SEQUENCE LISTING (8 sequences)

SEQ ID NO: 1 - DNA, 24 bp, artificial
  COX1 forward primer
ctatacctattattcggcgcatga

SEQ ID NO: 2 - DNA, 20 bp, artificial
  COX1 reverse primer
cagctcggctcgaataagga

SEQ ID NO: 3 - DNA, 21 bp, artificial
  COX4 forward primer
gccatgttcttcatcggtttc

SEQ ID NO: 4 - DNA, 22 bp, artificial
  COX4 reverse primer
ggccgtacacatagtgcttctg

SEQ ID NO: 5 - DNA, 20 bp, artificial
  18S forward primer
ggacatctaagggcatcaca

SEQ ID NO: 6 - DNA, 20 bp, artificial
  18S reverse primer
aggaattgacggaagggcac

SEQ ID NO: 7 - DNA, 20 bp, artificial
  GAPDH forward primer
caacgaatttggctacagca

SEQ ID NO: 8 - DNA, 20 bp, artificial
  GAPDH reverse primer
aggggtctacatggcaactg

(19) United States
(12) Patent Application Publication
(10) Pub. No.: US 2023/0201168 A1
(43) Pub. Date: Jun. 29, 2023

(54) NEW THERAPY FOR THE TREATMENT OF TUMORS

(71) Applicants: Università degli Studi di Trento, Trento (IT); Fondazione Giovanni Celeghin - ONLUS, Pernumia (PD) (IT)

(72) Inventors: Alessandro Quattrone, Trento (IT); Ines Mancini, Povo (Trento) (IT); Denise Sighel, Albiano (Trento) (IT); Angelika Modelska, Marseille (FR)

(21) Appl. No.: 17/927,571

(22) PCT Filed: May 24, 2021
(86) PCT No.: PCT/EP2021/063765

§ 371 Date: Nov. 23, 2022

(30) Foreign Application Priority Data
May 27, 2020 (IT) 102020000012601

(51) Int. Cl. A61K 31/424; A61K 38/12; A61P 35/00; A61P 25/00

(52) U.S. Cl. CPC A61K 31/424; A61K 38/12; A61P 35/00; A61P 25/00

(57) ABSTRACT

Compound of formula (I) or pharmaceutically acceptable stereoisomers and/or salts thereof, alone or in combination with quinupristin or pharmaceutically acceptable salts thereof, for use in the treatment of tumors,

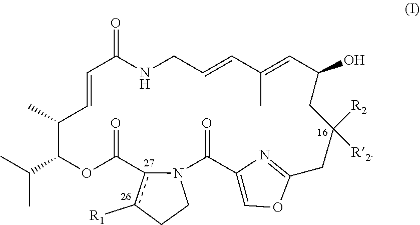

### FIELD OF THE INVENTION

The present description concerns a new therapy for the treatment of tumors.

### BACKGROUND

Glioblastoma multiforme (GBM) is the most common malignant primary brain tumor. Despite extensive treatment involving surgery, radiotherapy and chemotherapy, the survival of GBM patients remains extremely poor. The search for new effective therapies has proven very challenging throughout the last decades, with the only available drug for newly diagnosed GBM, temozolomide (TMZ), increasing the median patient survival from 12.1 to 14.6 months¹. This struggle is, at least partially, due to the presence of glioblastoma stem cells (GSCs), which are responsible for resistance to standard treatments as well as disease recurrence. Therefore, further research is much needed to identify novel effective therapeutics targeting GSCs.

For decades, tumor metabolism was believed to heavily rely on aerobic glycolysis, a phenomenon known as the “Warburg effect” thought to be caused by a decreased or damaged oxidative phosphorylation (OXPHOS). However, in the past years several studies have demonstrated that mitochondria and OXPHOS play an essential role in tumorigenesis and tumor progression. Rho⁰ cells, which are devoid of any mitochondrial DNA, undergo a dramatic reduction in tumorigenic potential and tend to acquire the mitochondrial DNA from neighboring normal cells to rescue tumorigenesis^{2,3}. Furthermore, OXPHOS remains the major source of ATP in many cancer types, even in the presence of enhanced rates of glycolysis⁴.

A growing body of literature reports that cancer stem cells (CSCs), including GSCs^{5,6}, substantially rely on OXPHOS for their energetic demands. Moreover, chemotherapeutic genotoxic drugs induce a shift in cancer cell metabolism towards upregulated OXPHOS and mitochondrial biogenesis.

The strong dependence of drug-tolerant CSC populations on OXPHOS suggests inhibition of mitochondrial metabolism as a new therapeutic avenue in oncology. A key supporting finding is that the anti-diabetic drug metformin has anti-cancer activity via reversible inhibition of the respiratory complex I. Other compounds possessing anticancer activity both in vitro and in vivo such as fenofibrate, lonidamide, atovaquone, and arsenic trioxide were also demonstrated to inhibit specific OXPHOS complexes, and some of them are under clinical evaluation. In GBM, inhibition of OXPHOS, but not glycolysis, abolishes GSC clonogenicity⁵. Moreover, the gene fusion of FGFR3-TACC3, present in 3% of the GBM cases, activates OXPHOS and mitochondrial biogenesis, and confers increased sensitivity to inhibitors of OXPHOS in vitro.

The assembly of OXPHOS complexes in the inner mitochondrial membrane is largely orchestrated by mitochondrial ribosomes (mitoribosomes) that synthesize thirteen transmembrane proteins. The mitochondrial translation machinery is upregulated in a subset of human tumors, and breast CSCs show metabolic reliance on mitoribosome synthesized proteins. Since human mitoribosomes, being descendants of bacterial ribosomes, differ from their cytosolic counterparts, they could be in principle selectively targeted to inhibit energy production.

There is therefore the need for the identification of compounds useful for the treatment of tumors, more specifically of tumors addicted to OXPHOS.

### SUMMARY OF THE INVENTION

The object of this disclosure is to provide a new therapy for the treatment of tumors.

According to the invention, the above object is achieved thanks to the subject matter recalled specifically in the ensuing claims, which are understood as forming an integral part of this disclosure.

The present invention concerns a compound of formula (I) and a pharmaceutical composition comprising the same, alone or in combination with quinupristin, for use in the treatment of a tumor,

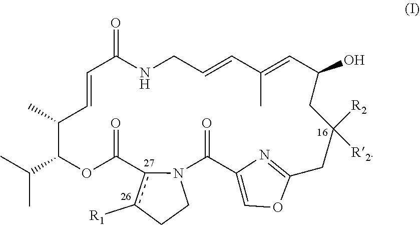

wherein

if the [symbol] bond represents a double bond, then R₁ is H, R₂ is selected from OH, F, NH₂, NHCH₃, N(CH₃)₂, OCF₃, Br, Cl, I, N₃, CN, SCN, and R′₂ is H, or R₂ and R′₂ form together an oxo group;

if the [symbol] bond represents a single bond with H in 27 (27R configuration), then R₁ is H, R₂ is selected from OH, F, NH₂, NHCH₃, N (CH₃)₂, OCF₃, Br, Cl, I, N₃, CN, SCN, and R′₂ is H, or R₂ and R′₂ form together an oxo group;

if the [symbol] bond represents a single bond with H in 27 (27S configuration), then R₁ is SO₂(CH₂)₂N(CH₂CH₃)₂ (26R configuration), R₂ is OH, and R′₂ is H, or R₂ and R′₂ form together an oxo group; and pharmaceutically acceptable stereoisomers and/or salts thereof.

### BRIEF DESCRIPTION OF THE DRAWINGS

The invention will now be described in detail, purely by way of an illustrative and non-limiting example and, with reference to the accompanying drawings, wherein:

FIG. 1: The chemical structures of quinupristin and the streptogramins A derivatives.

FIG. 2: The chemical structures of quinupristin/dalfopristin (30:70 w/w).

FIG. 3: Streptogramins A inhibit the growth of COMI GSCs line, alone or in combination with quinupristin. GI₅₀ values to several streptogramins A derivatives, alone or in combination with quinupristin, for COMI cells, n=3 biological replicates, mean±SD. D=dalfopristin, (16R)OH-D=(16R)-16-Deoxo-16-hydroxydalfopristin, (16S)OH-D=(165)-16-Deoxo-16-hydroxydalfopristin, PII_{A}=Pristinamycin II_{A}, (16R)OH-PII_{A}=(16R)-16-Deoxo-16-hydroxypristinamycin II_{A}, (16S)OH-PII_{A}=(16S)-16-Deoxo-16-hydroxypristinamycin II_{A}, (16R)F-PII_{A}=(16R)-16-Deoxo-16-fluoropristinamycin II_{A}, (16S)F-PII_{A}=(165)-16-Deoxo-16-fluoropristinamycin II_{A}, (16R)NHCH₃-PII_{A}=(16R)-16-Deoxo-16-methylaminopristinamycin II_{A}, (16S)NHCH₃-PII_{A}=(16S)-16-Deoxo-16-methylaminopristinamycin II_{A}, (16R)F-PII_{B}=(16R)-16-Deoxo-16-fluoropristinamycin II_{B}.

FIG. 4: Quinupristin/dalfopristin selectively inhibits the growth of GSCs at clinically relevant concentrations, is effective in hypoxic conditions and is more potent than TMZ. (a) GI₅₀ values of a panel of 21 GSC lines derived from 18 tumor samples at 48 and 72 h post-Q/D treatment, n=4, technical replicates. (b) Q/D GI₅₀ values for 8 GSC lines compared to Q/D GI₅₀ values for astrocytes derived from human fetal neural stem cells CB660 (Astrocytes) and for human lung fibroblasts MRC5 (Fibroblasts), n=3 biological replicates, mean±SD. (c) Representative immunofluorescence images for SOX2, NESTIN and GFAP staining on COMI, GB7 and VIPI cells grown in stemness and differentiation conditions. (d) Quantification of the normalized fluorescence intensity of NESTIN and GFAP immunostaining (left) and Q/D GI₅₀ values for COMI, GB7 and VIPI cells grown in stemness and differentiation conditions (right). For immunostaining quantification n=6000 objects for stem cells and n=1000 objects for differentiated cells, mean, ±SEM. The GI₅₀ values were calculated using n=4-7 biological replicates, mean±SD. (e) Viability of COMI and VIPI cells grown in normal and hypoxic conditions upon different doses of Q/D, n=4 technical replicates, mean±SD. Representative results of n=3 biological replicates. (f) Representative dose-response curves to Q/D and temozolomide (TMZ) for COMI and VIPI cells, n=3 biological replicates, mean±SD.

FIG. 5: Quinupristin/dalfopristin decreases clonogenicity, dysregulates cell cycle and promotes apoptosis. (a) Effects of Q/D treatment (1, 5 and 10 μM) on COMI cells grown in suspension. Example images from days 0, 4 and 9, scale bar 100 μm. (b) Sphere area quantification over the course of the nine-day experiment. Data were normalized on day 0. n=30 technical replicates, mean ±SEM. One representative experiment is shown, n=3 biological replicates. (c) Representative images of the gliomasphere formation assay, scale bar 500 μm. (d) The number of spheres greater than 100 μm was quantified, n=20 technical replicates, mean±SEM. ***p<0.001, unpaired two-tailed t-test. One representative result is shown, n=3 biological replicates. (e) Representative images of the effects of Q/D on the cell cycle in COMI cells assayed using EdU incorporation and PI staining. (f) Quantification of the percentage of cells in each phase of the cell cycle. n=3 biological replicates, mean±SD. *p<0.05 **p<0.01 ***p<0.001, unpaired two-tailed t-test. (g) Representative images of the induction of cell death via apoptosis upon treatment with Q/D in COMI cells as evaluated by Annexin V and PI staining. (h) Quantification of the percentage of Annexin V positive cells, n=6 biological replicates, mean±SD. **p<0.01, unpaired two-tailed t-test.

FIG. 6: Quinupristin/dalfopristin reduces the growth and invasion of GSC brain xenografts. (a) Assessment of blood-brain barrier (BBB) in brain xenografts of GFP-expressing GSC1. In the tumor core (upper panel), most of the microvessels lack continuous Glut1 staining (arrow), suggesting a disruption of BBB. In the periphery of the tumor (lower panel), microvessels with preserved BBB are frequently found (arrowheads). Scale bar, 25 μm. (b) Single tumor cells spreading along perivascular spaces of the BBB, which is either disrupt (arrow) or preserved (arrowhead). Scale bar, 150 μm. (c) Coronal brain sections of GFP-expressing GSC xenografts (upper panels), of control (non treated) mice or mice treated with Q/D (200 mg/kg i.p., three times a week for 3 weeks), scale bar 1000 μm. Details of brain regions invaded by GSCs (lower panels), scale bar 200 μm. (d) Number of GFP-expressing GSCs per high-power field in Q/D-treated and in control brain xenografts. n=10 non-superimposing 200× fields across the thalamus, fimbria, and optic tract of the right brain hemisphere, mean±SD. ***p<0.001, unpaired two-tailed t-test. (e) Histological examination of the liver, spleen, lung and kidney of mice treated with Q/D. (f) Kaplan-Meier survival analysis of Q/D treated mice (n=4) and control treated mice (n=4). Arrows represent treatment days (200 mg/kg i.p.). p=0.018, log rank test.

FIG. 7: Correlation analysis for 8 GSC lines of mitochondrial mass and viability after 48 h of Q/D treatment. Mitochondrial mass was assessed by immunofluorescence with COX4 antibody and analysis of the number of COX4 spots per area of cytoplasm. Viability was assessed after 48 h of Q/D treatment at 2.5, 5 and 10 μM. n=3-5 biological replicates, mean, ±SD. Correlation values (r) and p values were calculated using the Pearson correlation coefficient.

FIG. 8: Quinupristin/dalfopristin inhibits mitochondrial translation. (a) ³⁵S metabolic labeling assay on mitochondrial (left) and cytosolic (right) translation on COMI cells after 24 h treatment with Q/D. One representative result is shown, n=3 biological replicates. (b) Effects of increasing concentrations of Q/D on COX1, COX4 and β-tubulin proteins in COMI and VIPI cells after 48 h treatment. One representative result is shown, n=2 biological replicates. (c) Representative images of COX1 and COX4 immunofluorescence staining after Q/D treatment. (d) Quantification of COX1 and COX4 fluorescence intensity, n=3 technical replicates, mean±SD. **p<0.01, ***p<0.001, unpaired two-tailed t test. Representative results of n=3 biological replicates. (e) Effects of increasing concentrations of streptogramins A derivatives, alone or in combination with Q on COX1, COX4 and β-tubulin or α-actinin proteins in COMI cells after 48 h treatment. D=dalfopristin, (16R)OH-D=(16R)-16-Deoxo-16-hydroxydalfopristin, (16S)OH-D=(16S)-16-Deoxo-16-hydroxydalfopristin, PII_{A}=Pristinamycin II_{A}, (16R)OH-PII_{A}=(16R)-16-Deoxo hydroxypristinamycin II_{A}, (16S)OH-PII_{A}=(16S) Deoxo-16-hydroxypristinamycin II_{A}, (16R)F-PII_{A}=(16R)-16-Deoxo-16-fluoropristinamycin II_{A}, (16S)F-PII_{A}=(16S)-16-Deoxo-16-fluoropristinamycin II_{A}, (16R)F-PII_{B}=(16R)-16-Deoxo-16-fluoropristinamycin II_{B}.

FIG. 9: Quinupristin/dalfopristin inhibits mitochondrial translation and negatively affects OXPHOS functionality. (a) Effects of increasing concentrations of Q/D on COX1 and COX4 mRNA levels on COMI and VIPI cells after 48 h treatment. Data presented as fold change over control. n=4-5 biological replicates, mean±SD. Unpaired two-tailed t-test. (b) Immunostaining with anti-COX1 and anti-COX4 in Q/D-treated and in control brain xenografts, scale bar 20 μm. (c) Effects of Q/D on the functionality of OXPHOS complexes on COMI and VIPI cells after 48, 72 and 96 h of treatment. Coomassie staining serves as the loading control.

Representative results of n=2 biological replicates. (d) Oxygen consumption of COMI and VIPI cells upon treatment with Q/D for 48 h. Cells were evaluated for routine (R), complex I (CI), complex I and II (CI&II), uncoupled (ETS) and complex II (CII) respiration. Representative results of n=3 biological replicates. (e) Quantification of the changes in the mitochondrial membrane potential (MMP) as assessed by JC-1 staining in COMI cells. FCCP treatment was used as a positive control, n=4 biological replicates, mean±SD *p<0.05, unpaired two-tailed t-test as compared to non-treated. (f) Effects of increasing concentrations of Q/D on L-lactate production in COMI and VIPI cells after 48 h of Q/D treatment. The L-lactate levels were normalized on the number of cells, n=3 technical replicates, mean±SD. *p<0.05, **p<0.01, ***p<0.001, unpaired two-tailed t-test. Representative results of n=3 biological replicates.

FIG. 10: Mitochondrial translation inhibition and OXPHOS dysregulation upon quinupristin/dalfopristin treatment. (a) Effects of varying oxygen and Q/D concentrations on COX1, COX4 and β-tubulin protein expression on COMI (top) and VIPI (bottom) cells after 48 h of treatment. Representative results of n=2 biological replicates. (b) Immunoblotting detection of the large mitoribosomal subunit protein uL3m and the small mitoribosomal subunit protein uS17m after sucrose gradient sedimentation of COMI and HEK293 cell extracts after 2 h or 48 h treatment with Q/D. Immunoblotting signal was quantified using ImageLab software and presented in the graph. (c) Immunoblotting on blue native gels to assay for proteins belonging to complex I, V, IV and II. One representative result is shown (n=2 biological replicates).

### DETAILED DESCRIPTION OF THE INVENTION

In the description that follows, numerous specific details are given to provide a thorough understanding of the embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The headings provided herein are for convenience only and do not interpret the scope or meaning of the embodiments.

The present invention concerns, in different embodiments, a compound of formula (I) and a pharmaceutical composition comprising the compound of formula (I) for use in the treatment of a tumor,

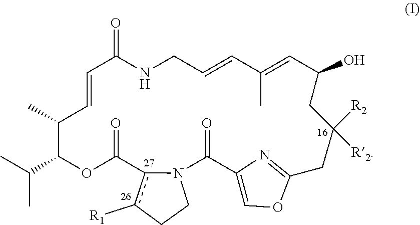

wherein

if the [symbol] bond represents a double bond, then R₁ is H, R₂ is selected from OH, F, NH₂, NHCH₃, N(CH₃)₂, OCF₃, Br, Cl, I, N₃, CN, SCN, and R′₂ is H, or R₂ and R′₂ form together an oxo group;

if the [symbol] bond represents a single bond with H in 27 (27R configuration), then R₁ is H; R₂ is selected from OH, F, NH₂, NHCH₃, N(CH₃)₂, OCF₃, Br, Cl, I, N₃, CN, SCN, and R′₂ is H; or R₂ and R′₂ form together an oxo group;

if the [symbol] bond represents a single bond with H in 27 (27S configuration), then R₁ is SO₂(CH₂)₂N(CH₂CH₃)₂ (26R configuration), R₂ is OH, and R′₂ is H, or R₂ and R′₂ form together an oxo group; and pharmaceutically acceptable stereoisomers and/or salts thereof.

In one or more embodiments, the compound of formula (I) is selected from the compounds listed in Table 1 below.

TABLE 1

| Structure | Name (and acronyms) |
|---|---|
| 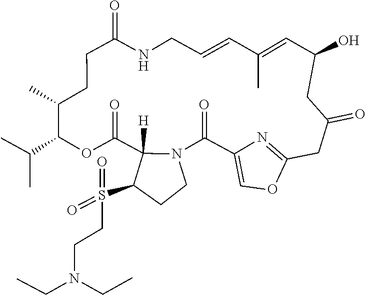 [SMILES: CCN(CC)CCS(=O)(=O)[C@@H]1CCN2C(=O)c3coc(n3)CC(=O)C[C@H](O)/C=C(C)/C=C/CNC(=O)CC[C@@H](C)[C@@H](C(C)C)OC(=O)[C@@H]12] | Dalfopristin (D) |
| 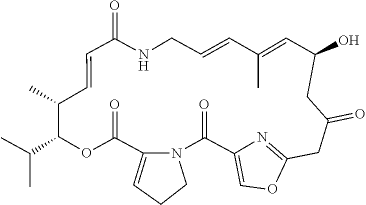 [SMILES: CC1=C\[C@@H](O)CC(=O)Cc2nc(co2)C(=O)N2CCC=C2C(=O)O[C@H](C(C)C)[C@H](C)/C=C/C(=O)NC\C=C\1] | Pristinamycin II_{A} (PII_{A}) |
| 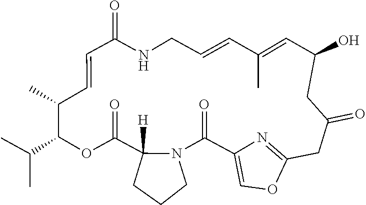 [SMILES: CC1=C\[C@@H](O)CC(=O)Cc2nc(co2)C(=O)N2CCC[C@@H]2C(=O)O[C@H](C(C)C)[C@H](C)/C=C/C(=O)NC\C=C\1] | Pristinamycin II_{B} (PII_{B}) |
| 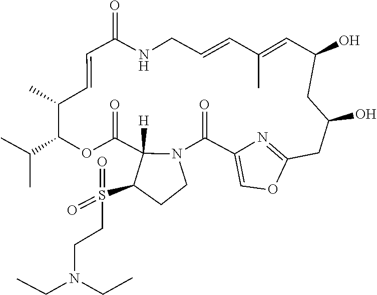 [SMILES: CCN(CC)CCS(=O)(=O)[C@@H]1CCN2C(=O)c3coc(n3)C[C@H](O)C[C@H](O)/C=C(C)/C=C/CNC(=O)/C=C/[C@@H](C)[C@@H](C(C)C)OC(=O)[C@@H]12] | (16R)-16-Deoxo-16- hydroxydalfopristin ((16R)OH-D) |
| 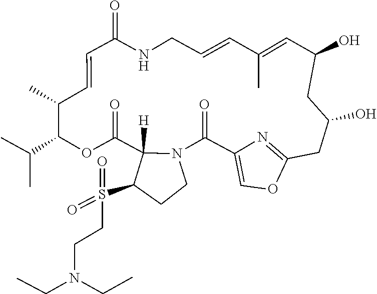 [SMILES: CCN(CC)CCS(=O)(=O)[C@@H]1CCN2C(=O)c3coc(n3)C[C@@H](O)C[C@H](O)/C=C(C)/C=C/CNC(=O)/C=C/[C@@H](C)[C@@H](C(C)C)OC(=O)[C@@H]12] | (16S)-16-Deoxo-16- hydroxydalfopristin ((16S)OH-D) |
| 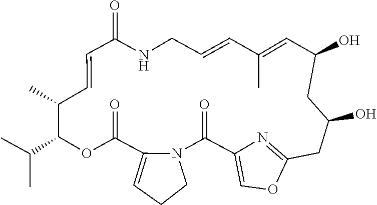 [SMILES: CC1=C\[C@@H](O)C[C@@H](O)Cc2nc(co2)C(=O)N2CCC=C2C(=O)O[C@H](C(C)C)[C@H](C)/C=C/C(=O)NC\C=C\1] | (16R)-16-Deoxo-16- hydroxypristinamycin II_{A} ((16R)OH-PII_{A}) |
| 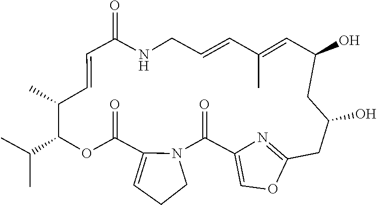 [SMILES: CC1=C\[C@@H](O)C[C@H](O)Cc2nc(co2)C(=O)N2CCC=C2C(=O)O[C@H](C(C)C)[C@H](C)/C=C/C(=O)NC\C=C\1] | (16S)-16-Deoxo-16- hydroxypristinamycin II_{A} ((16S)OH-PII_{A}) |
| 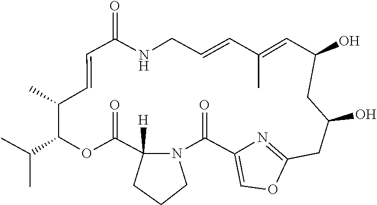 [SMILES: CC1=C\[C@@H](O)C[C@@H](O)Cc2nc(co2)C(=O)N2CCC[C@@H]2C(=O)O[C@H](C(C)C)[C@H](C)/C=C/C(=O)NC\C=C\1] | (16R)-16-Deoxo-16- hydroxypristinamycin II_{B} ((16R)OH-PII_{B}) |
| 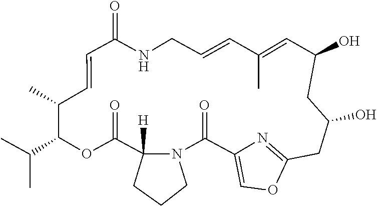 [SMILES: CC1=C\[C@@H](O)C[C@H](O)Cc2nc(co2)C(=O)N2CCC[C@@H]2C(=O)O[C@H](C(C)C)[C@H](C)/C=C/C(=O)NC\C=C\1] | (16S)-16-Deoxo-16- hydroxypristinamycin II_{B} ((16S)OH-PII_{B}) |
| 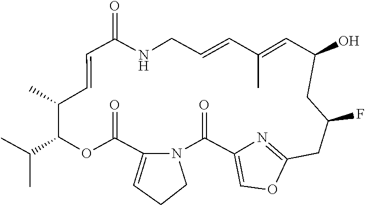 [SMILES: CC1=C\[C@@H](O)C[C@@H](F)Cc2nc(co2)C(=O)N2CCC=C2C(=O)O[C@H](C(C)C)[C@H](C)/C=C/C(=O)NC\C=C\1] | (16R)-16-Deoxo-16- fluoropristinamycin II_{A} ((16R)F-PII_{A}) |
| 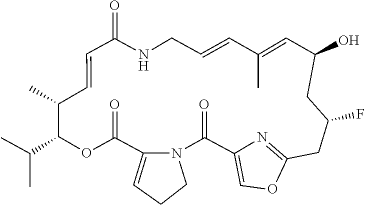 [SMILES: CC1=C\[C@@H](O)C[C@H](F)Cc2nc(co2)C(=O)N2CCC=C2C(=O)O[C@H](C(C)C)[C@H](C)/C=C/C(=O)NC\C=C\1] | (16S)-16-Deoxo-16- fluoropristinamycin II_{A} ((16S)F-PII_{A}) |
| 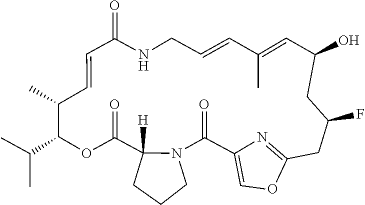 [SMILES: CC1=C\[C@@H](O)C[C@@H](F)Cc2nc(co2)C(=O)N2CCC[C@@H]2C(=O)O[C@H](C(C)C)[C@H](C)/C=C/C(=O)NC\C=C\1] | (16R)-16-Deoxo-16- fluoropristinamycin II_{B} ((16R)F-PII_{B}) |
| 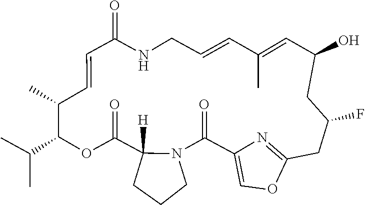 [SMILES: CC1=C\[C@@H](O)C[C@H](F)Cc2nc(co2)C(=O)N2CCC[C@@H]2C(=O)O[C@H](C(C)C)[C@H](C)/C=C/C(=O)NC\C=C\1] | (16S)-16-Deoxo-16- fluoropristinamycin II_{B} ((16S)F-PII_{B}) |
| 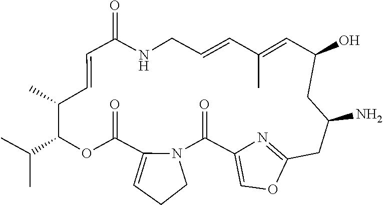 [SMILES: CC1=C\[C@@H](O)C[C@@H](N)Cc2nc(co2)C(=O)N2CCC=C2C(=O)O[C@H](C(C)C)[C@H](C)/C=C/C(=O)NC\C=C\1] | (16R)-16-Deoxo-16- aminopristinamycin II_{A} ((16R)NH₂-PII_{A}) |
| 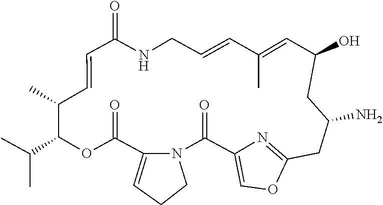 [SMILES: CC1=C\[C@@H](O)C[C@H](N)Cc2nc(co2)C(=O)N2CCC=C2C(=O)O[C@H](C(C)C)[C@H](C)/C=C/C(=O)NC\C=C\1] | (16S)-16-Deoxo-16- aminopristinamycin II_{A} ((16S)NH₂-PII_{A}) |
| 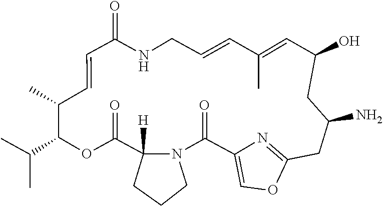 [SMILES: CC1=C\[C@@H](O)C[C@@H](N)Cc2nc(co2)C(=O)N2CCC[C@@H]2C(=O)O[C@H](C(C)C)[C@H](C)/C=C/C(=O)NC\C=C\1] | (16R)-16-Deoxo-16- aminopristinamycin II_{B} ((16R)NH₂-PII_{B}) |
| 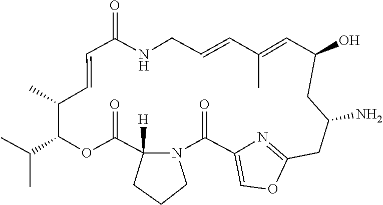 [SMILES: CC1=C\[C@@H](O)C[C@H](N)Cc2nc(co2)C(=O)N2CCC[C@@H]2C(=O)O[C@H](C(C)C)[C@H](C)/C=C/C(=O)NC\C=C\1] | (16S)-16-Deoxo-16- aminopristinamycin II_{B} ((16S)NH₂-PII_{B}) |
| 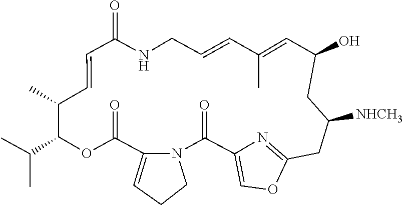 [SMILES: CN[C@H]1Cc2nc(co2)C(=O)N2CCC=C2C(=O)O[C@H](C(C)C)[C@H](C)/C=C/C(=O)NC/C=C/C(C)=C/[C@@H](O)C1] | (16R)-16-Deoxo-16- methylaminopristinamycin II_{A} ((16R)NHCH₃-PII_{A}) |
| 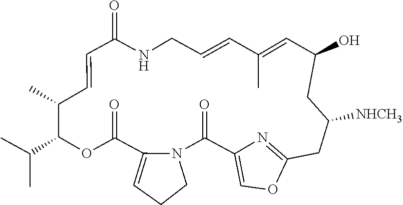 [SMILES: CN[C@@H]1Cc2nc(co2)C(=O)N2CCC=C2C(=O)O[C@H](C(C)C)[C@H](C)/C=C/C(=O)NC/C=C/C(C)=C/[C@@H](O)C1] | (16S)-16-Deoxo-16- methylaminopristinamycin II_{A} ((16S)NHCH₃-PII_{A}) |
| 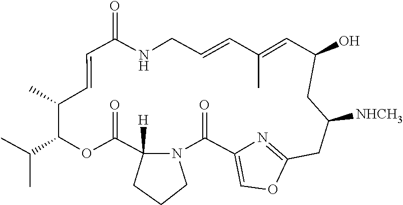 [SMILES: CN[C@H]1Cc2nc(co2)C(=O)N2CCC[C@@H]2C(=O)O[C@H](C(C)C)[C@H](C)/C=C/C(=O)NC/C=C/C(C)=C/[C@@H](O)C1] | (16R)-16-Deoxo-16- methylaminopristinamycin II_{B} ((16R)NHCH₃-PII_{B}) |
| 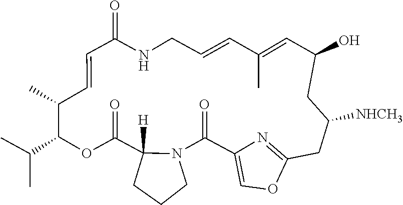 [SMILES: CN[C@@H]1Cc2nc(co2)C(=O)N2CCC[C@@H]2C(=O)O[C@H](C(C)C)[C@H](C)/C=C/C(=O)NC/C=C/C(C)=C/[C@@H](O)C1] | (16S)-16-Deoxo-16- methylaminopristinamycin II_{B} ((16S)NHCH₃-PII_{B}) |
| 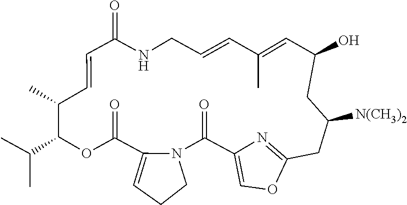 [SMILES: CC1=C\[C@@H](O)C[C@@H](N(C)C)Cc2nc(co2)C(=O)N2CCC=C2C(=O)O[C@H](C(C)C)[C@H](C)/C=C/C(=O)NC\C=C\1] | (16R)-16-Deoxo-16- dimethylaminopristinamycin II_{A} ((16R)N(CH₃)₂-PII_{A}) |
| 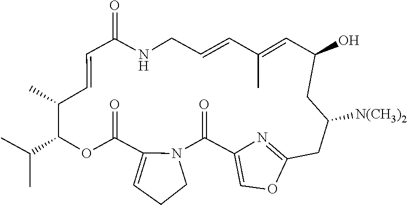 [SMILES: CC1=C\[C@@H](O)C[C@H](N(C)C)Cc2nc(co2)C(=O)N2CCC=C2C(=O)O[C@H](C(C)C)[C@H](C)/C=C/C(=O)NC\C=C\1] | (16S)-16-Deoxo-16- dimethylaminopristinamycin II_{A} ((16S)N(CH₃)₂-PII_{A}) |
| 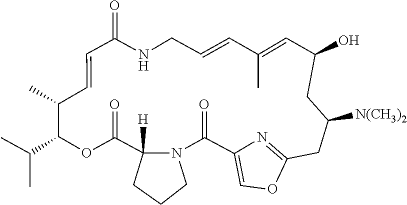 [SMILES: CC1=C\[C@@H](O)C[C@@H](N(C)C)Cc2nc(co2)C(=O)N2CCC[C@@H]2C(=O)O[C@H](C(C)C)[C@H](C)/C=C/C(=O)NC\C=C\1] | (16R)-16-Deoxo-16- dimethylaminopristinamycin II_{B} ((16R)N(CH₃)₂-PII_{B}) |
| 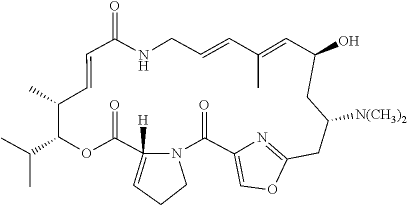 | (16S)-16-Deoxo-16- dimethylaminopristinamycin II_{B} ((16S)N(CH₃)₂-PII_{B}) |
| 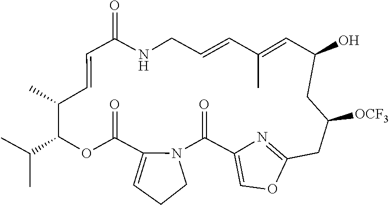 [SMILES: CC1=C\[C@@H](O)C[C@@H](OC(F)(F)F)Cc2nc(co2)C(=O)N2CCC=C2C(=O)O[C@H](C(C)C)[C@H](C)/C=C/C(=O)NC\C=C\1] | (16R)-16-Deoxo-16- trifluoromethoxypristinamycin II_{A} ((16R)OCF₃-PII_{A}) |
| 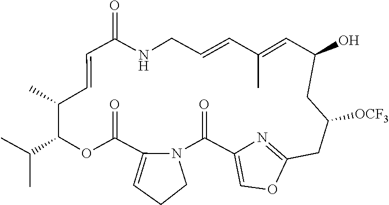 [SMILES: CC1=C\[C@@H](O)C[C@H](OC(F)(F)F)Cc2nc(co2)C(=O)N2CCC=C2C(=O)O[C@H](C(C)C)[C@H](C)/C=C/C(=O)NC\C=C\1] | (16S)-16-Deoxo-16- trifluoromethoxypristinamycin II_{A} ((16S)OCF₃-PII_{A}) |
| 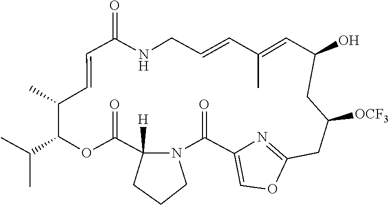 [SMILES: CC1=C\[C@@H](O)C[C@@H](OC(F)(F)F)Cc2nc(co2)C(=O)N2CCC[C@@H]2C(=O)O[C@H](C(C)C)[C@H](C)/C=C/C(=O)NC\C=C\1] | (16R)-16-Deoxo-16- trifluoromethoxypristinamycin II_{B} ((16R)OCF₃-PII_{B}) |
| 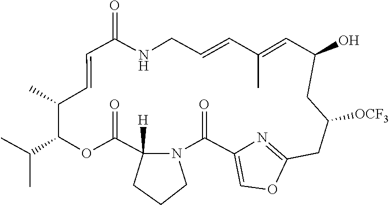 [SMILES: CC1=C\[C@@H](O)C[C@H](OC(F)(F)F)Cc2nc(co2)C(=O)N2CCC[C@@H]2C(=O)O[C@H](C(C)C)[C@H](C)/C=C/C(=O)NC\C=C\1] | (16S)-16-Deoxo-16- trifluoromethoxypristinamycin II_{B} ((16S)OCF₃-PII_{B}) |

In one or more embodiments, the compound of formula (I) is in combination with quinupristin or pharmaceutically acceptable salts thereof.

In one or more embodiments, R₂ is selected among OH, F and NHCH₃.

In one or more embodiments, R₂ and R′₂ form together an oxo group.

In a preferred embodiment, the compound of formula (I) is selected from:

(16R)-16-deoxo-16-hydroxypristinamycin II_{A},

(16S)-16-deoxo-16-hydroxypristinamycin II_{A},

(16R)-16-deoxo-16-dalfopristin,

(16S)-16-deoxo-16-dalfopristin,

(16R)-16-deoxo-16-fluoropristinamycin II_{A},

(16S)-16-deoxo-16-fluoropristinamycin II_{A}

(16R)-16-deoxo-16-fluoropristinamycin II_{B},

(16R)-16-Deoxo-16-methylaminopristinamycin II_{A},

(16S)-16-Deoxo-16-methylaminopristinamycin II_{A},

pristinamycin II_{A} and

dalfopristin.

In a further preferred embodiment, the compound of formula (I) is selected from:

(16R)-16-deoxo-16-hydroxypristinamycin II_{A},

(16R)-16-deoxo-16-dalfopristin,

(16R)-16-deoxo-16-fluoropristinamycin II_{A},

(16R)-16-deoxo-16-fluoropristinamycin II_{B},

(16R)-16-Deoxo-16-methylaminopristinamycin II_{A},

(16S)-16-Deoxo-16-methylaminopristinamycin II_{A}, pristinamycin II_{A} and dalfopristin.

In a preferred embodiment, the compound of formula (I) is selected from:

(16R)-16-deoxo-16-hydroxypristinamycin II_{A};

(16S)-16-deoxo-16-hydroxypristinamycin II_{A};

(16R)-16-deoxo-16-dalfopristin;

(16S)-16-deoxo-16-dalfopristin;

(16R)-16-deoxo-16-fluoropristinamycin II_{A};

(16S)-16-deoxo-16-fluoropristinamycin II_{A};

(16R)-16-deoxo-16-fluoropristinamycin II_{B};

(16R)-16-Deoxo-16-methylaminopristinamycin II_{A};

(16S)-16-Deoxo-16-methylaminopristinamycin II_{A};

pristinamycin II_{A}; and

odalfopristin;

and is in combination with quinupristin or pharmaceutically acceptable salts thereof.

In one or more embodiments, the combination of quinupristin and dalfopristin (or salts thereof) contains quinupristin and dalfopristin in a weight ratio equal to 30:70.

In one or more embodiments, the tumor is dependent on oxidative phosphorylation.

In one or more embodiments, the tumor is selected from glioblastoma multiforme, acute myeloid leukemia, chronic myeloid leukemia, epithelial ovarian cancer, pancreatic ductal adenocarcinoma, colorectal cancer, prostate cancer, melanoma, breast cancer and lung cancer.

In one or more embodiments, the tumor is glioblastoma multiforme.

In one or more embodiments, the compound of formula (I) is suitable for being administered along with at least one other treatment of the tumor. Preferably, the at least one other treatment of the tumor is selected from surgery, radiation, chemotherapy.

In one or more embodiments, the compound of formula (I) is suitable for intravenous administration.

In an embodiment, the present invention concerns a pharmaceutical composition comprising: (a) a compound of formula (I) or pharmaceutically acceptable stereoisomers and/or salts thereof (as disclosed above), alone or in combination with quinupristin or pharmaceutically acceptable salts thereof, and (b) a pharmaceutically acceptable carrier for use in the treatment of a tumor, preferably a tumor dependent on oxidative phosphorylation.

The pharmaceutical composition is for use in the treatment of a tumor selected from glioblastoma multiforme, acute myeloid leukemia, chronic myeloid leukemia, epithelial ovarian cancer, pancreatic ductal adenocarcinoma, colorectal cancer, prostate cancer, melanoma, breast cancer and lung cancer, preferably glioblastoma multiforme.

In an embodiment, the pharmaceutically acceptable carrier is an aqueous solution, preferably a water solution. Preferably, the water solution contains adjuncts, for example preservatives, stabilisers, wetting agents and/or emulsifiers, solubilizers, salts for regulating the osmotic pressures and/or buffers, as well as other adjuncts known in the art and commonly used. In the preferred embodiment, the water solution comprises dextrose, preferably at a concentration of about 5% (weight/volume). Preferably, the volume of the water solution is comprised between 250 mL and 500 mL.

In an embodiment, the compound of formula (I) (alone or in combination with quinupristin for use according to the present description) is in solid state, i.e. in the form of powder or lyophilized powder, and is dissolved in the pharmaceutically acceptable carrier above described right before being administered to a subject in need thereof, in order to obtain the pharmaceutical composition above described.

The present description also discloses a method for treating a tumor comprising administering to a patient in need thereof a compound of formula (I) as defined above or pharmaceutically acceptable salts thereof alone or in combination with quinupristin or pharmaceutically acceptable salts thereof in an amount sufficient to carry out said treatment.

The invention will now be described in detail, by way of non-limiting examples, with reference to compounds of formula (I) for use in the treatment of glioblastoma multiforme. It is clear that the scope of this description is in no way limited to the use in this specific tumor type, since the compounds of formula (I) described herein, alone or in combination with quinupristin, can be used in other types of tumors addicted to oxidative phosphorylation and can be administered alone or along with at least one other treatment of the tumor.

A growing body of literature suggests that targeting mitochondria, and in particular mitochondrial OXPHOS, in several types of CSCs results in destabilization of energy homeostasis, providing a new target for therapy. Currently, at least 14 inhibitors of one of the OXPHOS complexes have been evaluated in vivo or in clinic, while at least six are under preclinical testing, demonstrating that OXPHOS is an emerging target in oncology. Probably the most important example to date is the repurposing of the anti-diabetic drug metformin, which was shown to reversibly inhibit complex I, resulting in cytotoxic effects in different CSC types, including GSCs. Metformin is currently in more than three hundred ongoing clinical trials in combination with standard treatments.

In GBM high OXPHOS levels measured by complex IV activity are an independent negative prognostic factor. Two different small molecules can efficiently target complex IV function in GSCs both in vitro and in vivo. Very recently, IACS-010759, a potent small-molecule inhibitor of complex I, was also shown to inhibit proliferation in vitro and in vivo in GSCs reliant on OXPHOS.

OXPHOS can also be affected by selectively suppressing translation of the 13 proteins encoded by the mitochondrial DNA. The present inventors reasoned that by inhibiting mitochondrial translation the formation and functionality of the OXPHOS complexes I, III, IV and V could be hampered, leading to pronounced detrimental effects on the viability of GSCs. The feasibility of this strategy was demonstrated by the identification of tigecycline in acute myeloid leukemia stem cells, later shown to be effective in combination with the targeted drugs imatinib and venetoclax.

The inventors identified the streptogramins class of antibiotics as effective in GSCs growth inhibition. Streptogramins are a class of antibiotics consisting of a mixture of two structurally different compounds: the group A and the group B, which are known to act synergistically against bacteria. In detail, the inventors tested several streptogramins A derivatives, including dalfopristin (D), alone or in combination with quinupristin (Q), which belongs to streptogramin B group. All the derivatives were effective in inhibiting GSCs growth.

Notably, quinupristin/dalfopristin (Q/D) combination has been approved by the FDA for the treatment of persistent bacterial infections and could be easily repurposed for other uses. Therefore, the inventors further characterized Q/D effects on GSCs. They proved that Q/D decreased cell viability of GSCs grown both as adherent cultures and as gliomaspheres with unprecedented power and generality. In fact, the combination was effective on a large panel of GSCs and there was no correlation between the sensitivity and the molecular features of the cells or the variable clinical features of the patients from whom these lines were established, suggesting that Q/D could be used extensively on GBM patients. The suitability of Q/D repurposing in GBM is supported by the fact that the range of GI₅₀ values obtained in vitro matches blood concentration values achievable in patients treated with Q/D for bacterial infections. The inventors further explored the possibility of repurposing Q/D for GBM by demonstrating that Q/D preferentially targets GSCs rather than astrocytes or primary fibroblasts, suggesting a suitable therapeutic window. They also showed in vitro that GSCs are more sensitive to Q/D compared with their differentiated progeny, revealing a preferential GSC targeting for Q/D. It was also shown that the systemic administration of Q/D leads to cytotoxic effects on transplanted GSCs in vivo, significantly reducing the degree of tumor invasion in the brain and prolonging the survival of GBM-bearing mice.

Importantly for GBM therapy, Q/D can perfectly exert its cytotoxic effects and inhibit mitochondrial translation under hypoxic conditions. GBM tumors are largely hypoxic, and GSCs were identified in both GBM perivascular areas and hypoxic regions. Since drugs targeting the OXPHOS complexes are documented to alleviate or even eradicate tumor hypoxia, Q/D could exert an important, indirect antitumor effect in GBMs by promoting oxygenation and downregulating neoangiogenesis.

The present findings provide important insights also into the Q/D molecular mechanism of action. It was confirmed that Q/D binds to the mitoribosome and inhibits polypeptide synthesis, resulting in dysfunctional OXPHOS.

Like for any newly proposed anticancer drug, a major issue for favoring the transfer of Q/D to the clinical setting is the possibility to stratify patients. Some CSCs, including GSCs⁶, have been shown to present a certain degree of metabolic flexibility to inhibition of either glycolysis or OXPHOS, upregulating one of the two metabolic pathways in order to compensate for the inhibition of the other. GSCs from GBM patients bearing a homozygous deletion of the key glycolytic enzyme enolase 1 (ENO1) (3.3% of the cases) present a reduced capacity for compensatory glycolysis and thus an increased sensitivity to the complex I inhibitor IACS-010759. Other molecular alterations can produce a similar effect, for example, the FGFR3-TACC3 gene fusion (3% of GBM cases) confers special sensitivity to OXPHOS inhibitors by activating mitochondrial metabolism. Therefore, despite the fact that a marked variability in the GI₅₀ spectrum in GSCs from 18 different patients was not observed, a search for the more responsive patients to Q/D based on genome features is feasible, exploiting single genotypes as ENO1 loss-of-function or the FGFR3-TACC3 translocation, or more complex molecular signatures able to predict either high reliance on OXPHOS or glycolysis impairment.

Here it is described the identification of the streptogramins class as effective in inhibiting GSCs growth. Moreover, the inventors focused on the detailed characterization of Q/D action, a drug proposed for GBM therapy. Q/D acts via selective inhibition of mitochondrial translation and consequent OXPHOS dysregulation. It is extremely effective against GSCs both in vitro and in vivo models, displaying a potency over ten times that of TMZ. Notably, Q/D is an FDA-approved drug which achieves blood concentrations comparable to those necessary to inhibit GSC proliferation.

Results

### Streptogramins are Effective on GSCs

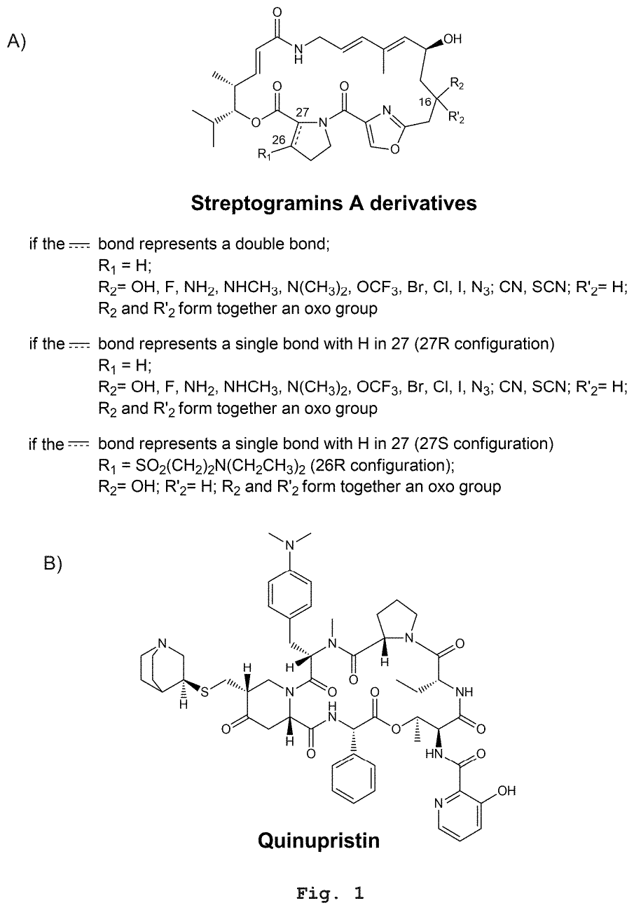

Streptogramins are a class of antibiotics consisting of a mixture of two structurally different compounds: the group A (also called M) streptogramins (or pristinamycin II), which are polyunsaturated cyclic peptolides, and the group B (also called S) streptogramins (or pristinamycin I), which are cyclic hexadepsipeptides. Streptogramins A and B are known to act synergistically against bacteria, and are used in combination in a fixed 70:30 (w/w) ratio, respectively. In detail, the inventors tested several streptogramins A derivatives, including dalfopristin (D), alone or in combination with quinupristin (Q), which belongs to streptogramin B group (FIG. 1). Notably, the Q/D (30:70 w/w) combination (FIG. 2) is an FDA-approved antibiotic for the treatment of skin infections and is traded as Synercid®.

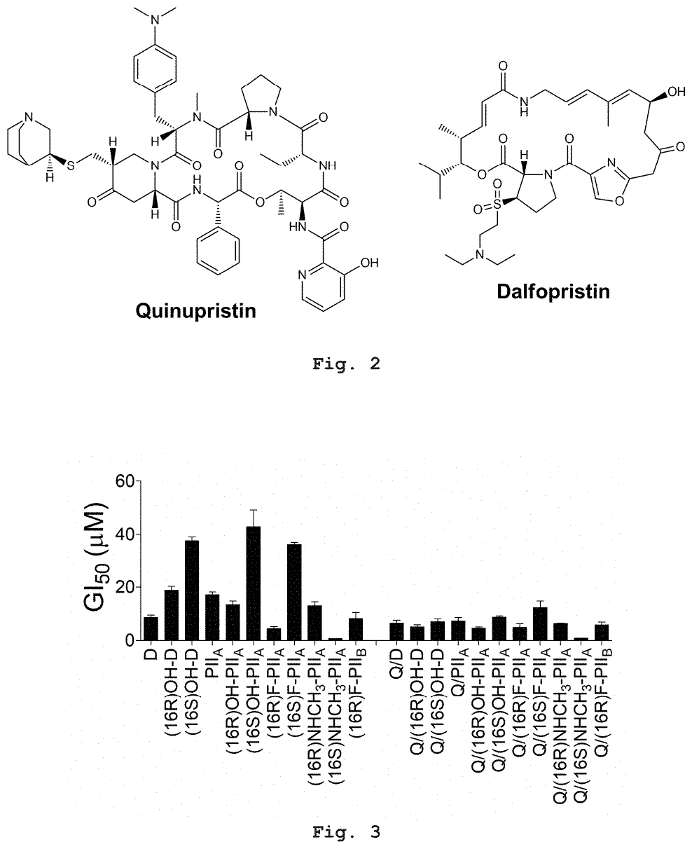

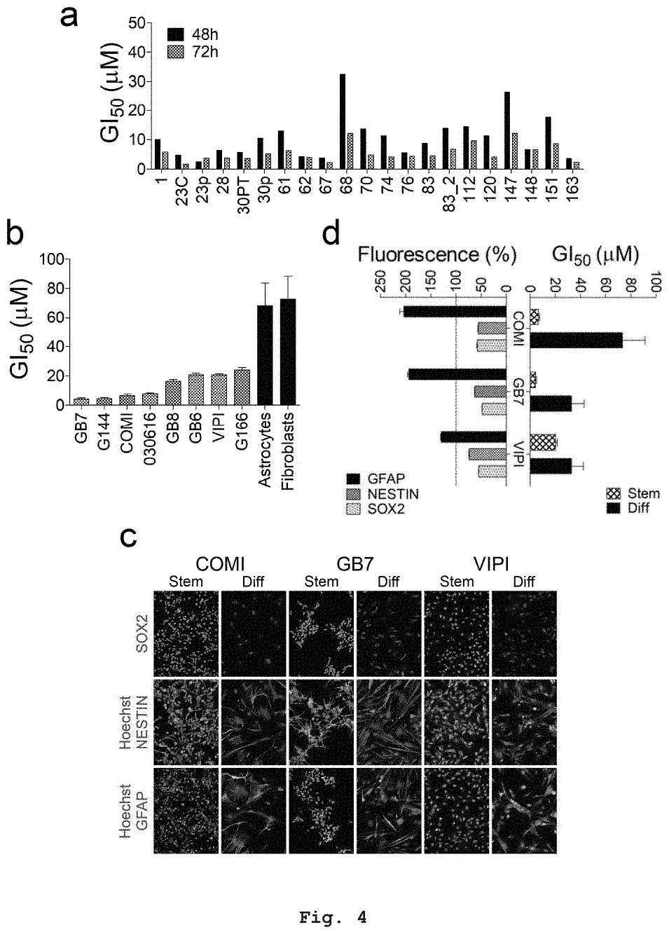

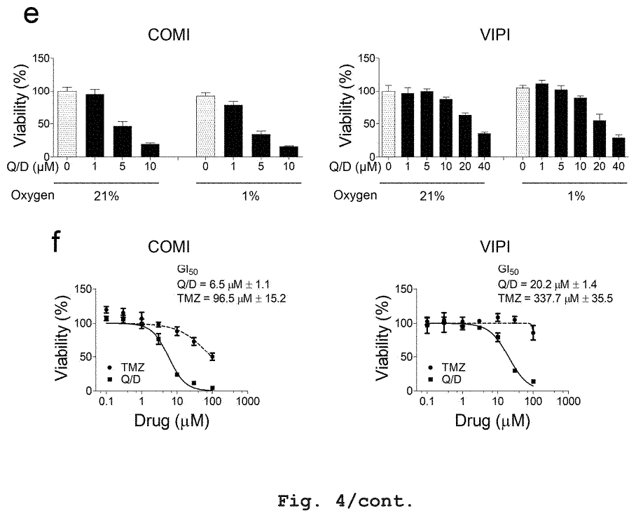

FIG. 3 reports the growth inhibition (GI50 values of the streptogramins A derivatives, alone or in combination with Q, on COMI GSC line.

### Quinupristin/dalfopristin Selectively Inhibits the Growth of GSCs at Clinically Relevant Concentrations

GBMs are characterized by inter- and intra-tumoral heterogeneity with GSCs known as responsible for drug resistance. Given that Q/D combination is an FDA-approved drug, it was decided to use it for further experiments. The activity of Q/D was assessed on a GSC panel composed of 21 lines derived from 18 patients with variable clinical features (Table 2, cell line characterization in Marziali et al., 2016⁷). The GIso values spanned from 2.5 to 32.5 μM after 48 h, narrowing to a range varying from 1.7 to 12.2 μM after 72 h of treatment (FIG. 4a). These values are in the range of the maximal blood concentration values achievable in patients treated with Q/D for bacterial infections, which varies between 14 and 32 μM (corresponding to 10.7 and 24.2 μg/mL) after an administration of 12.6 and 29.4 mg/kg, respectively. These results emphasize that the inhibitory effect of Q/D is time- and dose-dependent and is exerted at clinically relevant concentrations. In table 2: a) KPS=Karnofsky performance status; b) t DIS (mos)=disease time, from symptom onset to neurosurgery (months); c) PFS (mos)=progression free survival (months); d) OS (mos)=overall survival (months); e) PREOP RT=preoperative radiotherapy; f) DIM (cm)=dimension (cm); g) 5-ALA=5-aminolevulinic acid; h) MGMT=O⁶-methylguanine DNA-methyltransferase.

The inventors then attempted to correlate the Q/D sensitivity with clinical parameters of the patients from which these GSC lines were derived and with GBM markers, such as the EGFRvIII or PTEN status, but no statistically significant correlations emerged (Table 3). This negative result suggests that Q/D affects GSCs irrespective of the patient's clinical and basic molecular profile. In table 3, p-values for the correlation between primary cell features and clinical parameters of the patients from whom these GSC lines were derived and GI₅₀ values of the GSC lines at 48 h and 72 h of Q/D treatment are provided. The association of GIs₅₀ values with continuous features was computed by Pearson's correlation coefficient and with categorical ones by ANOVA test. In table 3: a) KPS=Karnofsky performance status; b) t DIS (mos)=disease time, from symptom onset to neurosurgery (months); c) PFS (mos)=progression free survival (months); d) OS (mos)=overall survival (months); e) PREOP RT=preoperative radiotherapy; f) DIM (cm)=dimension (cm); g) 5-ALA=5-aminolevulinic acid; h) MGMT=O⁶-methylguanine DNA-methyltransferase.

To assess the selectivity of Q/D for GSCs, the inventors evaluated its cytotoxicity in two normal diploid cells, astrocytes differentiated from a human fetal neural stem cell line (CB660) and a lung fibroblast cell line (MRC5), and compared it to the Q/D effects on another panel of 8 GSCs. The cells were treated with a range of Q/D concentrations and their viability assessed (FIG. 4b). The GI₅₀ values were 68.3±15.5 μM for the CB660-derived astrocytes and 72.7±15.7 μM for the MRCS cells, values substantially higher than the GI₅₀ of the other eight GSC lines tested.

The effect of Q/D treatment on the differentiated GSC progeny was then examined. To this aim, the inventors exposed three GSC lines to a pro-differentiation environment by culturing them in media deprived of growth factors and supplemented with 10% FBS for 14 days, and finally assessed their status by checking the expression of stemness (SOX2 and NESTIN) and differentiation (GFAP) markers by immunofluorescence (FIG. 4c). COMI and GB7 cells differentiated to a greater extent than VIPI, as evidenced by a higher increase in the expression of GFAP and decrease of SOX2 and NESTIN at the end of the treatment (FIG. 4d, left). The GI₅₀ of the differentiated COMI and GB7 cells were strikingly higher than their stem counterpart, while the less differentiated VIPI cells showed a much smaller increase in sensitivity to Q/D than their stem counterpart. This suggests that well differentiated GSCs are less sensitive to Q/D treatment (FIG. 4d, right).

Taken together, these results indicate that Q/D has a universal and selective inhibitory effect on GSC growth at clinically achievable concentrations.

TABLE 2

| | AGE | | | t DIS^{b} | PFS^{c} | OS^{d} | | PREOP | |
|---|---|---|---|---|---|---|---|---|---|
| GSC | (ys) | SEX | KPS^{a} | (mos) | (mos) | (mos) | RECURRENCE | RT^{e} | LOCALIZATION |
| 23p | 77 | M | 80 | 2 | 1 | 2 | no | no | parietal |
| 163 | 56 | M | 60 | 5 | 1 | 2 | no | no | parietal |
| 67 | 48 | M | 20 | 2.5 | 1 | 2 | no | no | parietal |
| 62 | 64 | M | 80 | 36 | 10 | 14 | yes | yes | frontal |
| 23c | 77 | M | 80 | 2 | 1 | 2 | no | no | parietal |
| 76 | 48 | F | 70 | 2.5 | 11 | 16 | no | no | frontal |
| 30PT | 44 | M | 70 | 1 | 5 | 7.5 | no | no | frontal |
| 28 | 72 | M | 90 | 1.5 | 6 | 11.5 | no | no | frontal |
| 148 | 55 | M | 50 | 6 | 5 | 8 | yes | yes | parietal |
| 83 | 52 | M | 70 | 0.5 | 3 | 8 | no | no | temporal |
| 1 | 40 | M | 80 | 2.5 | 6 | 12.5 | no | no | temporal |
| 30P | 44 | M | 70 | 1 | 5 | 7.5 | no | no | frontal |
| 74 | 70 | F | 60 | 1.5 | 2 | 8 | no | no | frontal |
| 120 | 53 | M | 80 | 25 | 8 | 16.5 | yes | yes | parietal |
| 61 | 59 | M | 80 | 2 | 3 | 6 | no | no | occipital |
| 70 | 67 | F | 60 | 2.5 | 6 | 9 | no | no | parietal |
| 83_2 | 52 | M | 70 | 0.5 | 3 | 8 | no | no | temporal |
| 112 | 49 | F | 70 | 2.5 | 3 | 6 | no | no | parietal |
| 151 | 69 | M | 80 | 4 | 60 | 72 | no | no | occipital |
| 147 | 69 | F | 60 | 2 | 7 | 11 | no | no | frontal |
| 68 | 58 | M | 60 | 3 | 4 | 10.5 | no | no | parietal |

| | DIM^{f} | TOTAL | | | | | | Ki67 |
|---|---|---|---|---|---|---|---|---|
| GSC | (CM) | RESECTION | 5-ALA^{g} | MGMT^{h} | EGFRvIII | PTEN | VEGF | (%) |
| 23p | 5 | yes | no | UM | neg | normal | iper | 50 |
| 163 | 4.6 | yes | no | UM | neg | ipo | normal | 15 |
| 67 | NA | yes | no | UM | neg | ipo | iper | 20 |
| 62 | NA | yes | no | M | neg | normal | iper | 10 |
| 23c | 5 | yes | no | UM | neg | normal | iper | 50 |
| 76 | 3.5 | yes | no | UM | neg | NA | iper | 15 |
| 30PT | 3.7 | yes | no | M | pos | normal | iper | 10 |
| 28 | 5 | yes | yes | M | neg | ipo | iper | 5 |
| 148 | 4 | yes | no | UM | neg | normal | iper | 70 |
| 83 | 5 | yes | no | UM | pos | normal | iper | 40 |
| 1 | 7 | no | no | M | neg | normal | iper | 20 |
| 30P | 3.7 | yes | no | M | pos | normal | iper | 10 |
| 74 | NA | no | no | UM | pos | normal | iper | 15 |
| 120 | 2.6 | yes | no | UM | neg | normal | iper | 30 |
| 61 | 5 | yes | no | UM | pos | normal | normal | 35 |
| 70 | 1.6 | yes | no | UM | pos | ipo | iper | 20 |
| 83_2 | 5 | yes | no | UM | pos | normal | iper | 40 |
| 112 | 4.5 | yes | no | M | pos | ipo | iper | 18 |
| 151 | 4 | yes | no | M | neg | ipo | iper | 30 |
| 147 | 7 | yes | no | UM | neg | normal | iper | 25 |
| 68 | NA | yes | no | UM | neg | normal | normal | 10 |

TABLE 3

| | AGE | | | t DIS^{b} | PFS^{c} | OS^{d} | | PREOP | |
|---|---|---|---|---|---|---|---|---|---|
| GSC | (ys) | SEX | KPS^{a} | (mos) | (mos) | (mos) | RECURRENCE | RT^{e} | LOCALIZATION |
| GI₅₀ | 0.58 | 0.31 | 0.92 | 0.47 | 0.39 | 0.29 | 0.39 | 0.39 | 0.89 |
| 48 h | | | | | | | | | |
| GI₅₀ | 0.72 | 0.28 | 0.88 | 0.46 | 0.30 | 0.26 | 0.60 | 0.60 | 0.88 |
| 72 h | | | | | | | | | |

| | DIM^{f} | TOTAL | | | | | | Ki67 |
|---|---|---|---|---|---|---|---|---|
| GSC | (CM) | RESECTION | 5-ALA^{g} | MGMT^{h} | EGFRvIII | PTEN | VEGF | (%) |
| GI₅₀ | 0.44 | 0.93 | 0.58 | 0.73 | 0.87 | 0.59 | 0.22 | 0.54 |
| 48 h | | | | | | | | |
| GI₅₀ | 0.17 | 0.72 | 0.79 | 0.79 | 0.90 | 0.58 | 0.48 | 0.90 |
| 72 h | | | | | | | | |

Quinupristin/Dalfopristin is Active in Hypoxic Conditions and is More Effective than Temozolomide

GSCs are known to reside in dedicated tumor niches, i.e. anatomical regions defined by a unique microenvironment which preserves them in low oxygen and represses their differentiation. Despite their enrichment in these hypoxic niches, GSCs are still expected to be OXPHOS-reliant in vivo, since it was shown that 1% oxygen is sufficient for their OXPHOS⁵. In this context, the activity of Q/D on GSCs grown under normoxia and hypoxia (21% and 1%) was evaluated by assessing cell viability after Hoechst 33342 and PI staining (FIG. 4e). Under 1% O₂, viability of untreated cells was not affected and the cells still responded to Q/D treatment in a dose-dependent manner, supporting Q/D efficacy also on GSCs in hypoxic conditions.

Since GSCs tend to be resistant to TMZ, the cytotoxicity of Q/D was compared with that of TMZ in COMI and VIPI cells (FIG. 4f). In COMI cells the GI₅₀ value for Q/D was 6.5±1.1 μM while that for TMZ was 96.5±15.2 μM; in VIPI cells the GI₅₀ value for Q/D was 20.2±1.4 μM while for TMZ was 337.7±35.5 μM. These experiments demonstrate that Q/D is 15 times more effective than TMZ in terms of growth inhibition of GSCs.

Therefore, Q/D is equally able to affect GSCs in normoxia and hypoxia and is over an order of magnitude more potent than TMZ.

### Quinupristin/Dalfopristin Decreases Clonogenicity, Blocks Cell Cycle Progression and Promotes Apoptosis

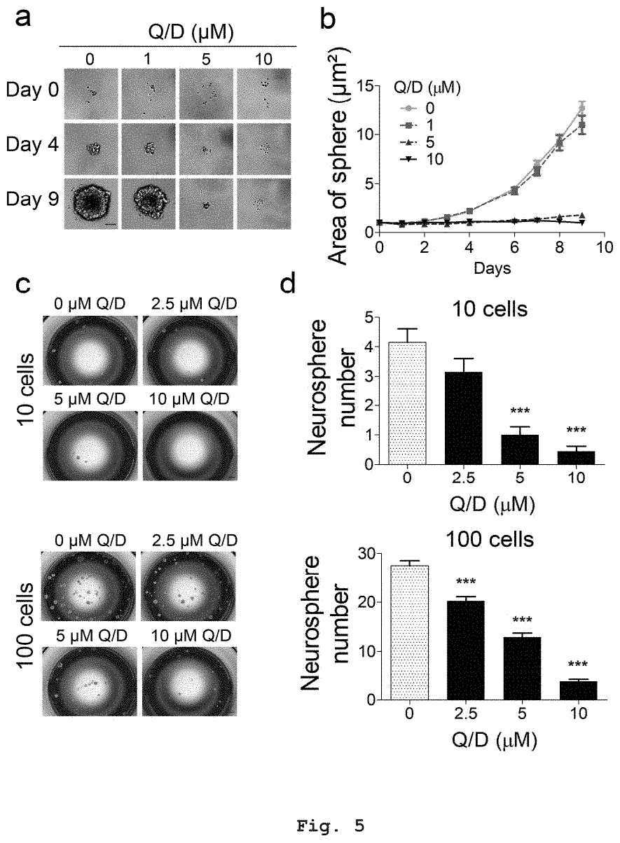

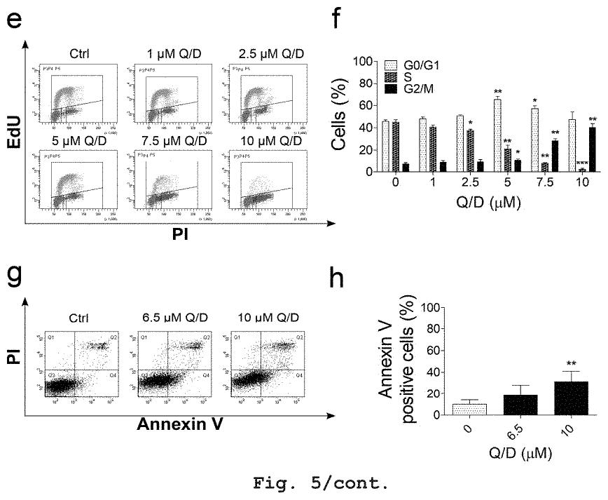

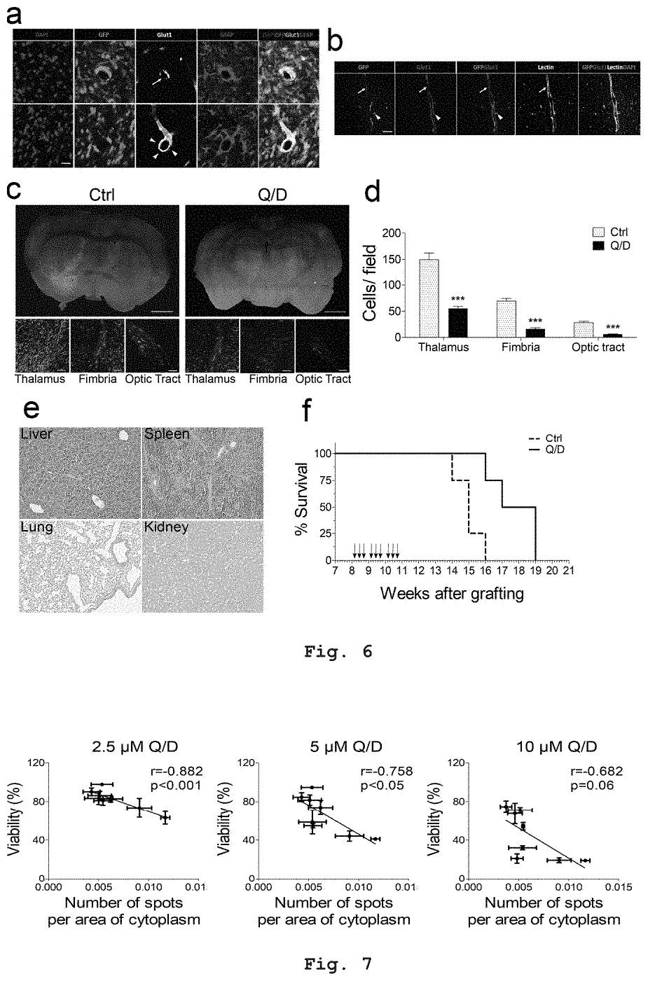

The inventors next investigated the extent of growth inhibition induced by Q/D in GSCs grown as gliomaspheres. Ten COMI cells per well were seeded in media with various Q/D concentrations and followed the formation of gliomaspheres over the course of nine days (FIGS. 5a, b). By measuring the area of the spheres, it was observed that the 1 μM Q/D treatment slightly inhibited gliomasphere formation, but that the 5 and 10 μM treatments, concentrations achievable in patients' blood, nearly completely abolished gliomasphere formation.

Given the profound inhibitory growth effect of Q/D on GSCs, the inventors wondered whether this drug could impact GSC self-renewal and clonogenic maintenance. To assess the effects of Q/D on GSC clonogenic potential, the gliomasphere forming ability of COMI cells after Q/D treatment was measured. COMI cells were grown in suspension with several concentrations of Q/D for 72 h, then the gliomaspheres were dissociated and seeded at a density of 10 or 100 cells per well in the absence of the drug. After 10 days the clonogenic potential was evaluated by counting the number of gliomaspheres reformed (FIGS. 5c, d). Q/D decreased the gliomasphere forming ability in a dose-dependent manner, confirming a substantial impact on GSC maintenance.

The functional effects induced on the GSC cell cycle were then investigated by performing EdU-PI staining upon treatment with Q/D, and by measuring the percentage of cells in each phase (FIGS. 5e, f). Increasing concentrations of Q/D led to a marked dose-dependent decrease in the number of cells in the S-phase, indicating inhibition of proliferation. In addition the inventors observed a significant increase in the number of cells in the G0/G1 phase at 5 μM Q/D treatment, indicating an accumulation of cells in this phase, followed by an increase in the number of cells in the G2/M starting at 5 μM and culminating at 10 μM Q/D treatments (FIG. 5f).

As Q/D decreases cell proliferation and GSC maintenance and dysregulates the cell cycle, the inventors next investigated whether Q/D induces apoptosis. A flow cytometric analysis was performed using Annexin V and PI staining and the percentage of apoptotic cells was estimated by calculating the percentage of Annexin V positive cells (FIGS. 5g, h). The treatment with 6.5 and 10 μM Q/D led to an increase in the percentage of apoptotic cells, even though the effect was significant only at 10 μM.

Overall, this phenotypic analysis of GSCs treated with Q/D in a range of 1-10 μM reveals a profound loss of clonogenic potential due to cell cycle arrest accompanied by apoptosis.

### Quinupristin/Dalfopristin Reduces the Growth and Invasion of GSC Brain Xenografts and Significantly Prolongs the SAurvival of GBM-Bearing Mice

Having observed the effectiveness of Q/D in vitro, the inventors decided to test whether it could induce similar effects in vivo using GSC mouse brain xenografts. In immunocompromised mice, human GSCs generate tumors that reproduce the histological and molecular features of the parent neoplasm and are resistant to chemotherapy. Tumor xenografts generated by intracerebral injection of human GSCs present a highly infiltrative GSC growth pattern that closely mimics the behavior of malignant human gliomas. A stable GFP-expressing GSC line (GFP-GSC1 line) was used, this cell line retaining the same in vitro sensitivity to Q/D as the parental primary GSC1 line and presenting a high propensity to invade the brain. In GFP-GSC1 brain xenografts in mice, the blood-brain barrier (BBB) was disrupted to different degrees, as assessed by immunofluorescence using anti-Glut1 antibody. However, vascular structures with preserved BBB were also detected in brain regions invaded by tumor cells (FIGS. 6a, b), recapitulating what happens in humans.

Using this mouse xenograft model, the effect of systemic administration of Q/D (i.p., three times a week for three weeks) on tumor growth was evaluated. Control mice (n=4) harbored tumors that invaded the homolateral striatum, piriform cortex, corpus callosum, anterior commissure, internal capsule, optic tract, septal nuclei, fimbria and hippocampus (FIG. 6c, left). In Q/D-treated mice (n=4; FIG. 6c, right), these brain regions were also populated by tumor cells, however, the degree of brain invasion was dramatically reduced, as demonstrated by the significant reduction in the density of tumor cells in the thalamus, fimbria, and optic tract (FIG. 6d). The histology of different organs (liver, spleen, lung and kidney) was also examined to evaluate the presence of Q/D-induced systemic toxicity, detecting only a small vacuolation of hepatocytes around the arteriole and central vein of hepatic lobules in the liver of Q/D treated mice (FIG. 6e). Using the same mouse xenograft model, the effect of systemic administration of Q/D (i.p., three times a week for three weeks) on mice survival was evaluated. Based on a Kaplan-Meier survival analysis, mice treated with Q/D (n=4) survived significantly longer than control mice (n=4) (FIG. 6f).

Taken together, these results show that Q/D administered by i.p. injection is able to cross the partially compromised BBB and affects the growth of GSCs in vivo, potently reducing the degree of invasion in the brain and significantly prolonging the survival of GBM-bearing mice.

### Quinupristin/Dalfopristin Inhibits Mitochondrial Translation Leading to OXPHOS Dysregulation

Having thoroughly assessed the ability of Q/D to induce an effective GSC clonal suppression in vitro and in vivo, the inventors moved to the investigation of its molecular mechanism of action. They first investigated whether the relative quantity of mitochondrial mass could be functionally related to the Q/D effectiveness in different GSC lines. They thus assessed the mitochondrial mass of 8 GSC lines, and then they treated them with different concentrations of Q/D for 48 h and correlated the susceptibility to Q/D treatment to their mitochondrial mass (FIG. 7). The data show that the viability after Q/D treatment is negatively correlated to the mitochondrial mass at 2.5 μM (r=−0.882, p<0.001), 5 μM (r=−0.758, p<0.05) and 10 μM (r=−0.682, p=0.06) treatments, indicating that GSCs with more mitochondria are more susceptible to Q/D-induced death.

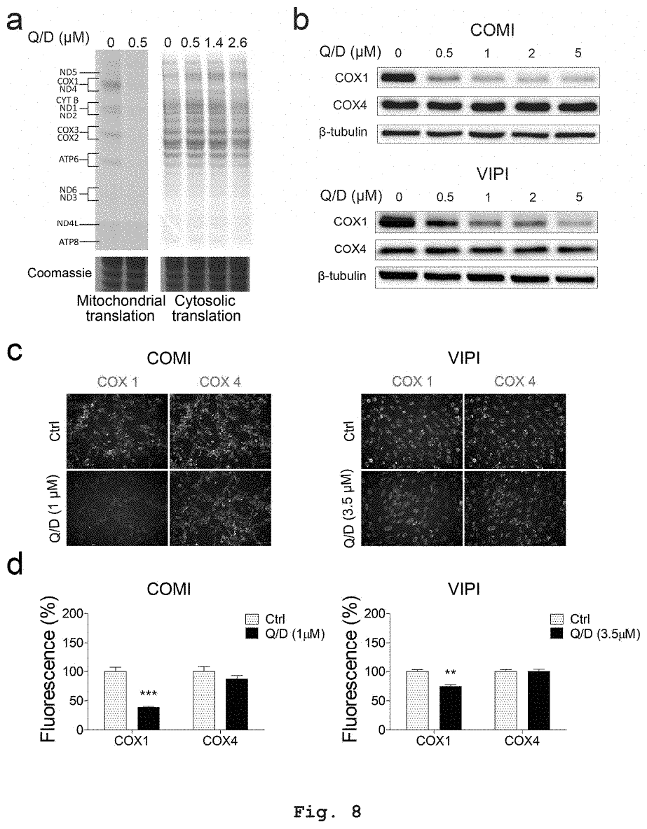

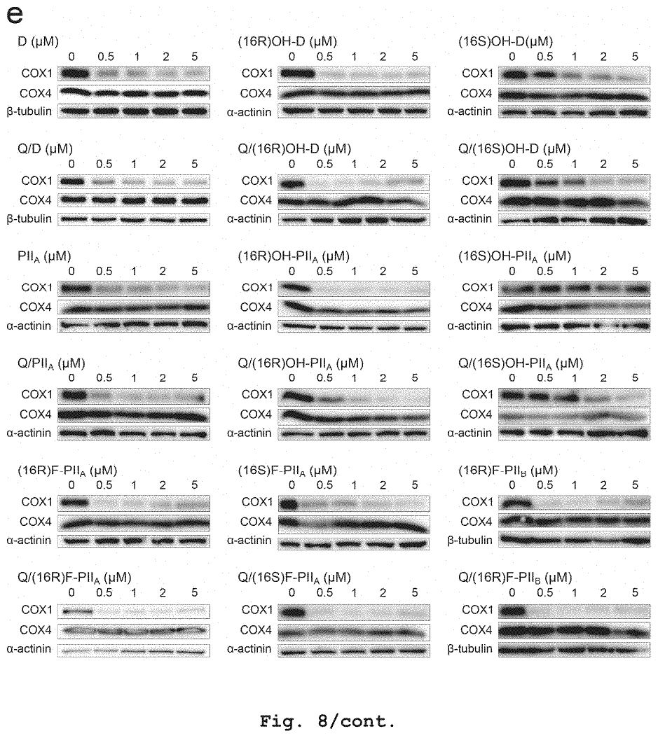

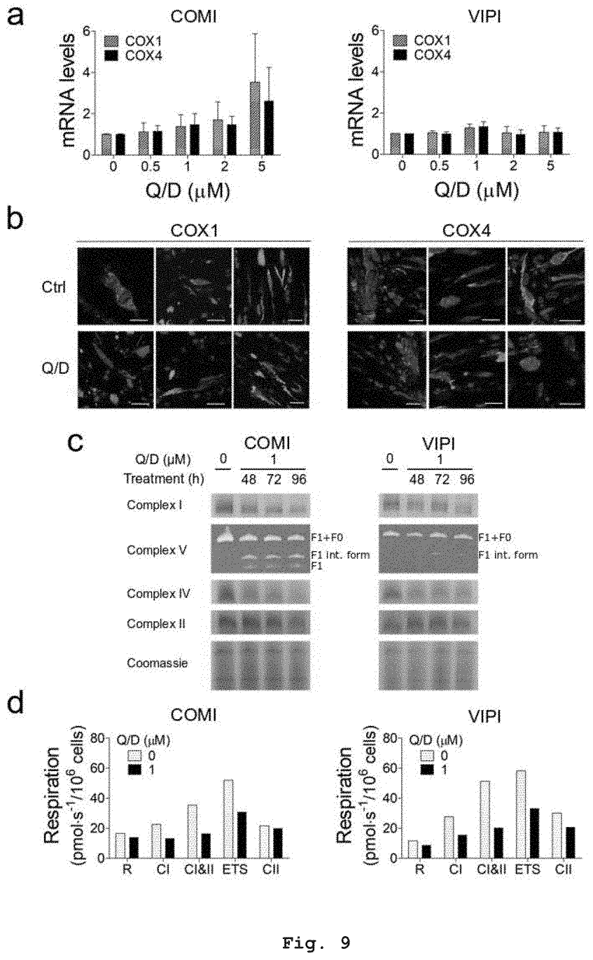

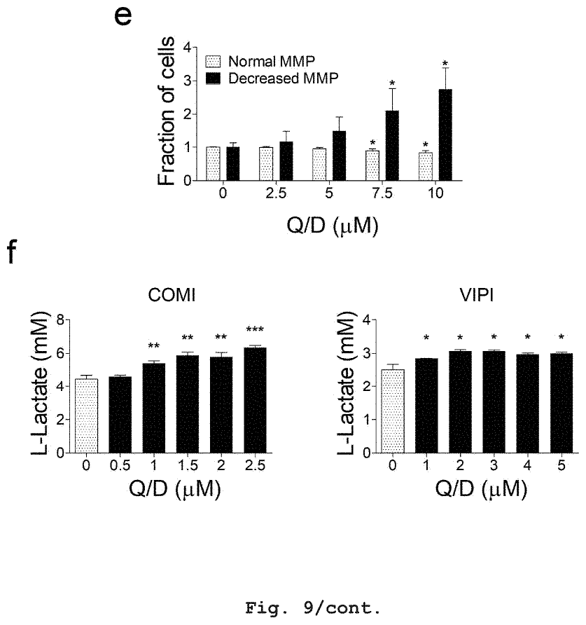

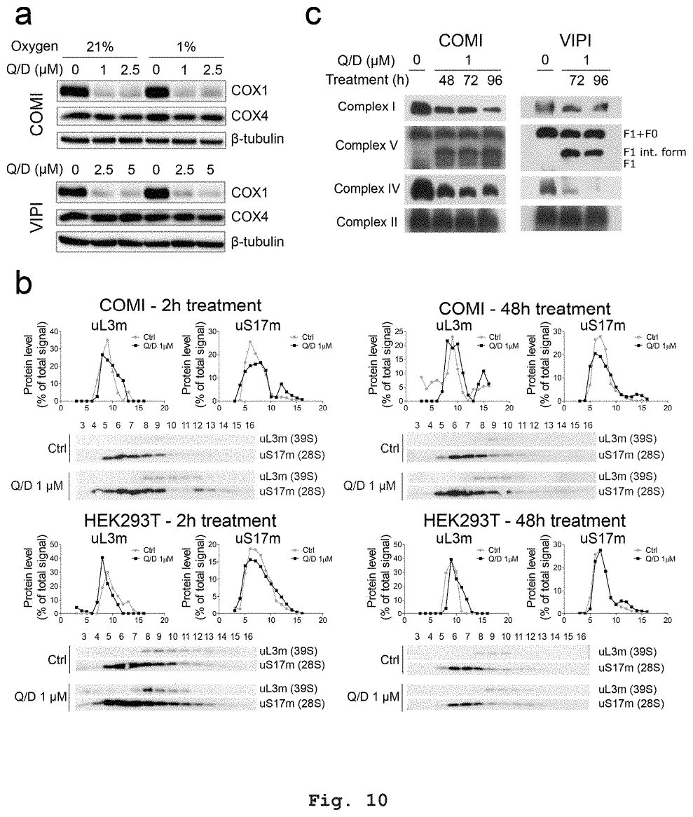

As a bacterial antibiotic, Q/D exerts its activity by inhibiting the bacterial ribosome, thus preventing protein synthesis. Given the evolutionary similarity between the functional core of the bacterial and human mitochondrial ribosomes, the inventors evaluated the effects induced by Q/D on mitochondrial translation and OXPHOS functionality. To determine whether Q/D specifically affects mitoribosomal function, they assayed for de novo synthesized proteins by mitochondrial or cytosolic ribosomes. To this end, metabolic labeling with ³⁵S-methionine on COMI cells treated with Q/D for 24 h was conducted (FIG. 8a). Q/D was very effective in inhibiting mitochondrial translation and nearly completely abolished it at 0.5 μM concentration. Importantly, no effects on cytosolic translation were noted at this or even higher Q/D concentrations (up to 2.6 μM tested). The effects on proteins synthesized in the mitochondria and cytosol were then confirmed by performing immunoblotting and immunofluorescence analysis on COMI and VIPI cells treated with Q/D for 48 h (FIGS. 8b-d). Cytochrome c oxidase subunit I (COX1) is synthesized in the mitochondria, whereas cytochrome c oxidase subunit IV (COX4) and β-tubulin are translated in the cytosol. The expression of COX1 was markedly decreased upon treatment with Q/D, whereas the expression of COX4 and β-tubulin remained unchanged. The same results were observed also when cells were treated with streptogramins A derivatives, alone or in combination with quinupristin (FIG. 8e). The decrease in COX1 protein levels upon Q/D treatment was confirmed also at 1% O₂ (FIG. 10a), suggesting that at 1% O₂ Q/D behaves in the same manner as at 21%. The effects of Q/D on mRNAs encoding COX1 and COX4 were then assessed but the inventors did not observe any significant changes (FIG. 9a). Therefore, Q/D acts specifically on mitochondrial translation.

The inventors next investigated whether Q/D treatment in vivo influenced the expression of COX1 and COX4 proteins in the same way as in vitro. Immunofluorescence staining with antibodies against COX1 and COX4 was performed, and the analysis showed that the level of COX1 was substantially reduced in tumor cells of Q/D treated mice as compared with vehicle-treated controls (FIG. 9b). The tumor cells invading the fimbria had the lowest expression of COX1. Conversely, the levels of COX4 were the same in control and Q/D treated mice, confirming the selective inhibition of mitochondrial translation also in vivo.

To investigate whether Q/D causes dissociation of mitoribosomal subunits or only translational stalling, the inventors performed sucrose gradient sedimentation of COMI and HEK293 cell extracts after 2 h or 48 h treatment with Q/D, followed by detection of the large mitoribosomal subunit protein uL3m and the small mitoribosomal subunit protein uS17m by immunoblotting. No shifts in the distribution were observed (FIG. 10b), indicating that Q/D binding does not cause the dissociation of the mitoribosomal subunits.

Since the thirteen proteins synthesized by mitoribosomes are an essential part of the OXPHOS complexes, the effects of Q/D on the functionality of these complexes were tested by performing blue native polyacrylamide gel electrophoresis (BN-PAGE) followed by in-gel activity assays on COMI and VIPI cells. The activity of complex I, IV and V, which are composed of proteins both mitochondrial and cytosolic in origin, was decreased upon Q/D treatment, whereas the activity of complex II, which is composed entirely of nuclearly encoded proteins, was unaffected (FIG. 9c). In parallel, the inventors assessed the amount of these complexes by BN-PAGE followed by immunoblotting, and they consistently found that the levels of complex I, IV and V, but not those of complex II, were decreased (FIG. 10c).

To verify whether the altered stoichiometry of the OXPHOS complexes led to a decreased mitochondrial respiratory capacity upon treatment with Q/D, mitochondrial respiration was tested using high-resolution respirometry. For both COMI and VIPI cells the functionality of complex I and complex II was decreased upon 1 μM Q/D treatment, even if this did not apparently impact on the basal respiration capacity (R). When the inventors measured the maximal oxygen consumption (ETS) through injection of the FCCP uncoupler, they found that it was decreased, indicating a substantial loss of reserve respiratory capacity (FIG. 9d).

Since functional OXPHOS is necessary to maintain the mitochondrial membrane potential (MMP), the inventors investigated whether Q/D induced a consequent loss in the MMP. They used the JC-1 dye to stain cells treated with Q/D at various concentrations and analyzed them by flow cytometry. In this experimental setting mitochondrial depolarization is indicated by a shift from red to green fluorescence. They determined the percentage of cells with normal MMP (having high red fluorescence and low green fluorescence), as well as the percentage of cells with lower MMP (having low red fluorescence and high green fluorescence), and subsequently quantified these changes (FIG. 9e). The inventors observed little changes upon 2.5 μM Q/D treatment, while from 5 μM onwards the percentage of cells with disrupted MMP increased in a dose-dependent manner, suggesting that indeed Q/D affects the MMP.

Another process by which cells can fulfill their ATP requirements is glycolysis, and switching to glycolysis is a potential mechanism of bioenergetic flexibility for OXPHOS targeted drugs in cancer cells. To verify whether upon treatment with Q/D GSCs switch to glycolysis, the levels of L-lactate, a product of the glycolytic pathway, were measured. A small but significant increase in L-lactate production starting from 1 μM Q/D treatment for both COMI and VIPI cells was observed (FIG. 9f), indicating a tendency to glycolytic switch.

Overall, these results clearly demonstrate that Q/D acts selectively by interfering with mitochondrial translation and consequently decreasing OXPHOS chain functionality in GSCs.

### Methods

### Cell Culture

Human glioblastoma stem cell lines COMI, VIPI, whose isolation and characterization have been previously reported⁸⁻¹⁰, were a kind gift from Dr. Antonio Daga (Azienda Ospedaliera Universitaria San Martino di Genova, Italy). These lines derive from two male patients diagnosed with grade IV GBM and have both been classified as belonging to the RTK II (Classic) cluster according to their EGFR amplification¹⁰. Biological grouping performed by Vecchio et al. was based on correlations with mutational status and DNA copy number variations of the main genes, TP53, IDH1, H3F3A, EGFR, PDGFRA and CDKN2A, which characterize the subgroups proposed by Sturm et al.¹¹ Table 4 reports COMI and VIPI biological group characterization performed by Vecchio and colleagues¹⁰.

TABLE 4

| CELLS | Biological Group | TP53 | IDH1 | H3F3A | EGFR | PDGFRA | CDKN2A |
|---|---|---|---|---|---|---|---|
| COMI | RTK II (Classic) | wt | wt | wt | Ampl | wt | Del |
| VIPI | RTK II (Classic) | mut | wt | wt | Ampl | wt | Del |

Wt: wild type; mut: mutation or copy number variations; ampl: genes locus amplification; del: gene deletions.
TP53 gene is considered mutated when either copy number changes or mutations are present. Adapted from¹⁰.

Human glioblastoma stem cell lines 030616 was a kind gift from Rossella Galli (San Raffaele Hospital, Milan, Italy).

Human glioblastoma stem cell lines COMI, VIPI and 030616 were cultured in DMEM/F-12 and Neurobasal media (1:1 ratio), supplemented with GlutaMAX (2 mM; Thermo Fisher Scientific), B27 supplement (1%; Thermo Fisher Scientific), Penicillin G (100 U/mL; Sigma Aldrich), bFGF (10 ng/mL; R&D Systems), EGF (20 ng/mL; R&D Systems) and heparin (2 μg/mL; Sigma Aldrich) at 37° C., 5% CO₂. Cells were grown either as spheres in suspension or as adherent cultures on laminin-coated flasks where they maintain intact self-renewal capacity¹².

Human glioblastoma stem cell lines GB6, GB7¹³, GB8, G144¹², G166¹² and the human fetal neural stem cell line CB660¹⁴ were a kind gift from Luciano Conti (CIBIO, University of Trento, Italy) and were cultured as adherent cultures on laminin-coated flasks in Euromed-N media (Euroclone), supplemented with GlutaMax (2 mM), B27 supplement (2%), N2 (1%; Thermo Fisher Scientific), Penicillin G (100 U/mL), bFGF (20 ng/mL), and EGF (20 ng/mL) at 37° C. 5% CO₂. Culture flasks were coated with laminin (10 μg/mL final, Thermo Fisher Scientific, cat. 23017015) and incubated for 3 h at 37° C. or overnight at 4° C. prior to use.

Human glioblastoma stem cell lines GSC23p, GSC163, GSC67, GSC62, GSC23C, GSC76, GSC3OPT, GSC28, GSC148, GSC83, GSC1, GSC30p, GSC74, GSC120, GSC61, GSC70, GSC83_2, GSC112, GSC151, GSC147 and GSC68, whose characterization has been reported in Marziali et al., 2016⁷, were a kind gift of Lucia Ricci-Vitiani (Istituto Superiore di Sanità, Italy) (Table 2 shows cell line characterization). These cell lines were grown as gliomaspheres in suspension in DMEMF12 serum-free medium containing 2 mM L-glutamine, 0.6% glucose, 9.6 mg/mL putrescine, 6.3 ng/mL progesterone, 5.2 ng/mL sodium selenite, 0.025 mg/mL insulin, 0.1 mg/mL transferrin sodium salt, in the presence of EGF (20 ng/mL), bFGF (10 ng/mL) and heparin (2 μg/mL) at 37° C., 5% CO₂.

Human Lung fibroblasts, MRCS (https://www.atcc.org/products/all/CCL-171.aspx), were cultured in EMEM media, supplemented with 10% FBS, GlutaMAX (2 mM) and Penicillin G (100 U/mL) at 37° C., 5% CO₂.

For GSCs differentiation, cells were grown on laminin-coated plates in the above media without growth factors and with the addition of 10% FBS for 14 days. For astrocyte differentiation, CB660 cells were grown on laminin-coated plates in the above media without growth factors and with the addition of 5% FBS for 3 weeks.

For hypoxia experiments, cells after treatment were grown in a hypoxic chamber (Invivo2 200, Baker Ruskinn, Hypoxic, 1% O₂).

### Compounds

Q/D combination was acquired from Santa Cruz Biotech. (cat. sc-391726, as mesylate complex), Dalfopristin (D) was acquired from Santa Cruz Biotech. (cat. sc-362728), Quinupristin (Q) was acquired from Bioaustralis (cat. BIA-Q1354).

Streptogramins A derivatives of formula (I) were synthesized as previously described i.a. in U.S. Pat. Nos. 5,242,938, 6,815,437, 6,541,451, 6,569,854, 7,166,594, 6,962,901, 7,232,799, GB2206879, Bacque et al., 2005¹⁵.

### Viability Assays

For the evaluation of the effect of Q/D, streptogramins A analogues (alone or in combination with Q) and TMZ on GSCs viability, cells were seeded into 96-well laminin-coated microtiter plates in 150 μL of media. The plates were incubated for 24 h prior to drug treatment. Serial drug dilutions were prepared in PBS to provide a total of seven drug concentrations plus control. 10 μL of these dilutions were added to each well, and the plates were incubated for additional 48 h. Each treatment was performed in technical quadruplicate. After the drug treatments, the cells were stained with Hoechst 33342 (1 μg/mL; Thermo Fisher Scientific, cat. H1399) and Propidium Iodide (PI, 1 μg/mL; Sigma Aldrich, cat. P4170) and incubated for 20 min shaking in the dark. The plates were then read using Operetta-High Content Imaging System (Perkin Elmer) and analyzed using the Harmony Software. The number of viable cells was calculated by subtracting PI positive cells from the total number of cells estimated by Hoechst 33342 staining and normalized on the non treated control.

For cytotoxicity analysis on GSCs grown as gliomaspheres, the cells were mechanically dissociated and plated in a 96-well plate, in triplicate.

Quinupristin/dalfopristin (Q/D) was added 24 h after cell plating. ATP levels were measured using the CellTiter-Glo® Luminescent Cell Viability Assay (Promega, cat. G7570) as per the manufacturer's instructions after 48 h and 72 h of treatment. Percentage viability was calculated upon normalization on the non treated control.

Dose-response curves were plotted and growth inhibition 50 (GI50) values calculated using the GraphPad Prism software.

### 3D Viability Assay

Ten cells/well were plated in Ultra-Low attachment round bottom 96 well plates (Costar) and treated with desired Q/D concentrations. The cells were centrifuged at 300 g for 30 sec, followed by the first acquisition using Operetta-High Content Imaging System (Perkin Elmer). The images were subsequently acquired over the course of 9-10 days. The area of the spheres formed was assessed using the Harmony Software.

### Gliomasphere Formation Assay

COMI cells grown in suspension were plated and treated with Q/D for 72 h. The spheres were then dissociated, cells counted and plated at a density of 10 or 100 cells/well in a 96 well plate without the drug. After 10 days, the spheres formed were stained with 1 μM Calcein AM (Thermo Fisher Scientific, cat. C3100MP) by incubating for 20 min at 37° C., after which the spheres were imaged using Operetta-High Content Imaging System (Perkin Elmer) and analyzed using the Harmony Software. Only spheres greater than 100 μm were quantified. The experiment was performed in a biological triplicate, with 20 technical replicates each.

### Cell Cycle Assay

Cells were plated 24 h prior to treatment and incubated with Q/D for additional 48 h. Cell cycle analysis was conducted using FACS (BD FACSCanto II) after staining with the Click-IT EdU Flow Cytometry Assay kit (Thermo Fisher Scientific, cat.C10634) according to the manufacturer's instructions. The experiment was performed in a biological triplicate. Data were processed by the BD FacsDIVA V8.0.1™ software.

### Apoptosis Assays

Apoptosis was assessed using FITC Annexin V Apoptosis Detection Kit I (BD Pharmingen, BD Biosciences, cat. 556547). Cells were plated 24 h prior to treatment and incubated with Q/D for further 48 h. 200,000 cells were stained according to the manufacturer's instructions and analyzed using FACS (BD FACSCanto II). Data were processed by BD FacsDIVA V8.0.1™ software.

### Intracranial Implantation of Glioma Stem-Like Cells (GSCs) in Immunocompromised Mice, Analysis of Brain Xenografts and Survival Analysis of GBM-Bearing Mice

Experiments involving animals were approved by the Ethical Committee of the Istituto Superiore di Sanità, Rome, Italy. NOD-SCID mice (4-6 weeks old; Charles River, Italy) were implanted intracranially with 2×10⁵ green fluorescence protein (GFP)-expressing GSC#1 cells resuspended in 5 μL of serum-free DMEM. For grafting, the mice were anesthetized with intraperitoneal injection of diazepam (2 mg/100 g) followed by intramuscular injection of ketamine (4 mg/100 g). Animal skulls were immobilized in a stereotactic head frame and a burr hole was made 2 mm right of the midline and 1 mm anterior to the coronal suture, and cells were slowly injected using the tip of a 10-μL Hamilton microsyringe placed at a depth of 3.5 mm from the dura. After grafting, the animals were kept under pathogen-free conditions in positive-pressure cabinets and observed daily for neurological signs. Beginning 8 weeks after implantation, the mice (n=8) were treated with Q/D (200 mg/kg i.p.) three times weekly for 3 weeks. Control animals (n=8) were treated with vehicle. Body weight was monitored weekly. For the analysis of GSC invasion in brain xenografts, one week after discontinuation of therapy, the mice were deeply anesthetized and transcardially perfused with 0.1× PBS (pH 7.4), then treated with 4% paraformaldehyde in 0.1× PBS. The brain was removed, stored in 30% sucrose buffer overnight at 4° C., and serially cryotomed at 40 μm on the coronal plane. Images were obtained with a Laser Scanning Confocal Microscope (IX81, Olympus Inc, Melville, N.Y.). In Q/D-treated (n=4) and control (n=4) xenografts, the density of tumor cells was assessed by counting the number of GFP-expressing GSCs in 10 non-superimposing 200× fields across the thalamus, fimbria, and optic tract of the right brain hemisphere. For immunofluorescence sections were incubated in ice-cold 100% methanol for 10 min at 20° C. for permeabilization. After a rinse in PBS for 5 min, sections were incubated in PBS containing 5% normal donkey serum for 45 min and then incubated in 1:1000 primary mouse anti-MTCO1 (Abcam, cod. 14705) or 1:500 rabbit anti-COX IV (Cell Signaling, cod. 4850) or 1:200 rabbit anti-Glucose Transporter-1 (Glut-1) (Merck Millipore, Burlington, Mass.) antibodies, overnight at 4° C. After rinses in PBS, sections were incubated for 1 h at room temperature with a 1:200 CY3 anti-mouse or anti-rabbit secondary antibodies (Vector Laboratories), respectively. Confocal images were generated using a Zeiss 510 Meta confocal microscope. For the analysis of mice survival, after the treatment the mice were kept under pathogen-free conditions in positive-pressure cabinets and observed daily for neurological signs. Body weight was monitored weekly. The mice were sacrificed when the body weight dropped to less than 80% of initial weight or at the appearance of neurological signs. Q/D treated mice n=4, control treated mice n=4.

### Mitochondrial Mass

Mitochondrial mass was assayed by staining the mitochondria with MitoTracker Orange (Thermo Fisher Scientific) or anti-COX4 antibody (Abcam) as described in the immunofluorescence section. The number of mitochondria was estimated by counting the number of MitoTracker Orange or COX4 positive spots per area of cytoplasm using Operetta-High Content Imaging System (Perkin Elmer) and analyzed by the Harmony Software.

### Mitochondrial and Cytosolic Protein Synthesis Assay

Cells were plated 24 h prior to treatment and incubated with Q/D for further 24 h prior to ³⁵S labelling. To assay for mitochondrial protein synthesis, growth medium was removed and cells were washed twice with methionine/cysteine-free DMEM medium, followed by an incubation in methionine/cysteine-free DMEM medium containing 96 pg/mL Cysteine, 1% B27 supplement, 1% GlutaMax; 1% Sodium Pyruvate, 10 ng/mL bFGF, 20 ng/mL EGF, 2 μg/mL heparin and 80 μg/mL emetine (Sigma Aldrich, cat. E2375) for 15 min at 37° C. Subsequently, [³⁵S]-methionine (Perkin Elmer, cat. NEG709A005MC) was added to a final concentration of 166.6 μCi/mL and the labeling was performed for 20 min at 37° C. The cells were then detached and pelleted at 4,000 rpm for 5 min. The pellet was washed three times with 1 mL of PBS. Cell pellets were resuspended in protein lysis buffer containing protease inhibitors and 1.25 U/μL benzonase. Protein concentrations were measured with Pierce™ BCA Protein Assay Kit (Thermo Fisher Scientific, cat. 23227) and equal amount of protein samples were separated on SDS-PAGE gels (NuPAGETM 12% Bis-Tris Protein Gels, Thermo Fisher Scientific, cat. NP0343BOX). The labelled proteins were visualized and quantified using a Phosphorlmager system and ImageQuant software (Molecular Dynamics, GE Healthcare). To assay for cytosolic translation, the above procedure was used without the addition of emetine.

### Immunoblotting

Total cell lysates were prepared from cells. Briefly, cells were washed with PBS and resuspended in lysis buffer (50 mM Tris-HCl pH 7.4, 150 mM NaCl, 1 mM EDTA, 0.25% NP-40, 0.1% Triton X-100, 0.1% SDS and supplemented with protease inhibitors). Protein concentrations were measured with PierceTM BCA Protein Assay Kit (Thermo Fisher Scientific, cat. 23227). Equal amounts of protein were separated on SDS-PAGE and transferred to nitrocellulose or PVDF (for anti-LC3 antibody only) membrane. Membranes were probed with anti-MTCO1 (COX1, Abcam, cat. ab14705), anti-COX4 (Cell Signaling, cat. 4850), anti-β-tubulin (Santa Cruz, cat. sc-53140), anti-LC3 (Cell Signaling, cat. 3868S), anti-MRPS17 (ProteinTech, cat. 18881-1-AP), anti-MRPL3 (Atlas Antibodies, cat. HPA043665) and secondary HRP-conjugated antibodies (Santa Cruz Biotechnology). Detection was performed using Amersham ECL Prime or Select Western Blotting Detection Reagent (GE Healthcare Life Sciences) and ChemiDoc Imaging System (Bio-Rad). Data were analyzed using ImageLab software.

### Immunofluorescence

The cells were fixed either with paraformaldehyde solution (4% v/v final, 15 min incubation at room temperature) or with 100% ice-cold methanol (5 min incubation at room temperature, only for LC3B IF), followed by two washes with PBS. The cells were then permeabilized with 0.3% Triton X-100-3% BSA in PBS for 45 min at room temperature. The incubation with the primary antibody was carried out at 4° C. overnight, followed by a wash with 3% BSA-PBS solution and incubation with the secondary antibody for 1 h at room temperature. Cell morphology was determined by staining with HCS CellMask™ Deep Red Stain (Thermo fisher Scientific, cat. H32721, 1:2000, 20 min, room temperature). The plates were then read either using Operetta-High Content Imaging System (Perkin Elmer) and analyzed by the Harmony Software or using the Leica TCS SP5 confocal microscope and processed by imaging softwares ImageJ (version v1.51w) and Photoshop. For the latter, z-stack images were acquired and LC3 puncta quantification was performed on image stacks of region of interests containing single cells, using the “3D Maxima finder” plugins of ImageJ. Both size and intensity threshold constraints were applied to the quantification.

### Confocal Imaging

Images were acquired on a Leica TCS SP5 confocal microscope with a 63× oil immersion objective, 2× zoom, 1024×1024 resolution, 200 Hz speed, lasers Argon 488 nm and Diode laser 633 nm, step 0.89 μm. Images were further analyzed and processed using imaging softwares ImageJ (Fiji) and Photoshop.

### RNA Extraction, Reverse Transcription and Quantitative Real-Time Polymerase Chain Reaction

Total RNA was extracted using QIAzol reagent

(QIAGEN) according to the manufacturer's instructions. Reverse transcription was performed using iScript Reverse Transcription Supermix (BioRad, cat. 170-8891) on C1000 Thermal Cycler (BioRad) and quantitative real-time PCR was performed using 2× qPCR SyGreen Mix Separate ROX (PCR Biosystems, cat. PB20.14-05) following the manufacturer's instructions on CFX384 Real-Time System (BioRad). All assays were performed in triplicate in 4-5 independent experiments. Data was analyzed using CFX Manager software (BioRad). Relative expression values of each target gene were normalized to GAPDH and 18S RNA level. The following primers were used at 500 nM concentration:

COX1:
5′- CTATACCTATTATTCGGCGCATGA-3′ (forward -
SEQ ID No.: 1)
and
5′-CAGCTCGGCTCGAATAAGGA -3′ (reverse -
SEQ ID No.: 2),
COX4:
5′ -GCCATGTTCTTCATCGGTTTC-3′ (forward - SEQ
ID No.: 3)
and
5′-GGCCGTACACATAGTGCTTCTG-3′ (reverse -
SEQ ID No.: 4),
18S:
5′-GGACATCTAAGGGCATCACA-3′ (forward - SEQ ID
No.: 5)
and
5′-AGGAATTGACGGAAGGGCAC-3′ (reverse - SEQ
ID No.: 6),
GAPDH:
5′-CAACGAATTTGGCTACAGCA-3′ (forward - SEQ
ID No.: 7)
and
5′-AGGGGTCTACATGGCAACTG-3′ (reverse -
SEQ ID No.: 8) .

### BN-PAGE and in Gel Complex Activity Assay

Cells were plated in two T75 flasks and treated with Q/D after 24 h for additional 48 h, 72 h and 96 h. Mitochondria were isolated in the following manner: cells were detached, pelleted and resuspended in 750 μL of MIB+BSA buffer (0.32 M sucrose, 1 mM EGTA pH 8, 20 mM Tris-HCl, pH 7.2, 0.1% fatty acid-free BSA). The cells were homogenized using Potter-Elvehjem homogeniser and centrifuged at 1,000g for 5 min at 4° C. The supernatant was collected and the pellet resuspended in MIB+BSA, rehomogenised and centrifuged again. The supernatant was collected and pooled with the first one, then centrifuged at 12,000 g for 10 min at 4° C. to pellet the mitochondria. The pellet of mitochondria was subsequently washed and resuspended in 100 μL of ACNA buffer (1.5 M aminocaproic acid, 50 mM BisTris, pH 7.00) and quantified using Qubit™ Protein Assay Kit (Thermo Fisher Scientific, cat. Q33212). Digitonin was added to a final concentration of 1% w/v, the samples were vortexed and incubated on ice for 20 min, followed by a centrifugation at 14,000 g for 30 min at 4° C. The supernatant was mixed with the loading buffer, and 50 μg of protein was separated on Blue Native PAGE gels (NativePAGE™ 3-12% BisTris Protein Gels, Thermo Fisher Scientific, cat. BN1001BOX). The gels were incubated overnight at room temperature with the respective complex substrates. Complex I: 2 mM Tris HCl, pH 7.4; 0.1 mg/mL NADH; 2.5 mg/mL iodonitrotetrazolium chloride. Complex II: 4.5 mM EDTA, 0.2 mM phenazine methosulfate, 84 mM succinic acid and 0.5 mg/mL iodonitrotetrazolium chloride. Complex IV: 0.5 mg/mL 3.3′-diamidobenzidine tetrahydrochloride (DAB), 50 mM phosphate buffer pH 7.4; 1 mg/mL cytochrome c, 0.2 M sucrose, 20 μg/mL (1 nM) catalase. Complex V: 3.76 mg/mL glycine, 5 mM MgCl₂, Triton X-100, 0.5 mg/mL lead nitrate, ATP, pH 8.4.

### Respiration Assay

High-resolution respirometry was performed using a 2 mL chamber OROBOROS Oxygraph-2k (Oroboros Instruments) at 37° C. Respiration rates were calculated as the negative time derivative of oxygen concentration measured in the closed respirometer and expressed per number of viable cells and corrected by residual oxygen consumption (ROX, after antimycin A addition). The amplified signal was recorded on a computer with online display of the calibrated oxygen concentration and oxygen flux (DatLab software for data acquisition and analysis; Oroboros Instruments). Cells were plated 24 h prior to treatment and incubated with Q/D for further 48 h. The cells were then detached and 1,000,000 cells were injected into each chamber. Oxygen consumption was evaluated for cellular ROUTINE respiration, and then cells were permeabilized with digitonin 4.1 uM in MiR05 medium (10 mM KH₂PO₄, 60 mM lactobionic acid, 20 mM HEPES, 3 mM MgCl₂, 0.5 mM EGTA, 20 mM taurine, 110 mM D-sucrose and 1 mg/mL BSA fatty acid free). Complex I activity was measured after malate (2 mM, glutamate (10 mM) and ADP (5 mM) injection, and complex I&II activity after additional succinate (10 mM) injection. The ETS capacity (maximum uncoupled respiration) was induced by stepwise titration of FCCP (typically 3-4 steps, 1 ul each of 1 mM FCCP). Complex II activity was measured after the addition of rotenone (0.5 uM). Residual respiration (ROX) was measured after inhibition with antimycin A (2.5 μM).

### Mitochondrial Membrane Potential Assessment

Cells were plated 24 h prior to treatment and incubated with Q/D for further 48 h. Positive control was treated with 100 uM FCCP for 10 min. Cells were detached and 500,000 cells were resuspended in fresh media containing 5 μg/mL JC-1 (Abcam, cat. ab113850), and incubated at 37° C. for 30 min. Cells were then centrifuged at 400 g for 5 min and resuspended in 0.5 mL PBS and analyzed using FACS (BD FACSCanto II). Data were processed by BD FacsDIVA V8.0.1™ software.

### Lactate Assay

Cells were plated in a 96 well plate and treated the following day with Q/D for 48 h. Media was collected and the lactate production was measured using Glycolysis Cell-Based Assay Kit (Cayman Chemical, cat. 600450) according to the manufacturer's instructions. The cells were then fixed with 4% v/v paraformaldehyde for 15 min at room temperature and stained with Hoechst 33342 (1 μg/mL). The nuclei were quantified using Operetta-High Content Imaging System (Perkin Elmer) and analyzed by the Harmony Software. The lactate production was normalized on the number of cells.

### Autophagy Assays

To assess for autophagic flux, cells were plated 24 h prior to treatment and incubated with 6.5 μM Q/D for further 48 h. In addition, for immunoblotting the cells were treated with 60 μM chloroquine for 24 h (Sigma Aldrich, cat. C6628), 6.5 nM bafilomycin for 3 h (Sigma Aldrich, cat. B1793) or 5 mM NH₄Cl for 3 h (Sigma Aldrich, cat. A9434). Immunoblotting was performed as described in the Immunoblotting section. LC3B-II was quantified by densitometric analysis (Image Lab 2.0.1 software, Biorad) and normalized on β-tubulin as a loading control. For immunofluorescence analysis, the cells were treated with 60 μM chloroquine, 6.5 nM bafilomycin or 10 mM NH₄Cl for 24 h. Immunofluorescence for LC3 staining was carried out according to the procedure described in the Immunofluorescence section. Cell morphology was determined with staining with CellMask Deep Red Stain (Thermo fisher Scientific, cat. H32721).

In order to evaluate the role of autophagy in Q/D cytotoxic activity, COMI cells were seeded into 96-well microtiter plates in 150 μL of media at plating densities of 4,000 cells/well. The plates were incubated for 24 h prior to drug treatment. Cells were pretreated with chloroquine (5, 10 and 20 μM) for 3 h and then treated with 6.5 μM of Q/D for further 48 h. The cells were stained with Hoechst 33342 (1 μg/mL; Thermo Fisher Scientific, cat. H1399) and Propidium Iodide (PI, 1 μg/mL; Sigma Aldrich, cat. P4170) and incubated for 20 min shaking in the dark. The plates were then read using Operetta-High Content Imaging System (Perkin Elmer) and analyzed using the Harmony Software. The number of viable cells was calculated by subtracting PI positive cells from the total number of cells estimated by Hoechst 33342 staining and normalized on the control. Each treatment was performed in technical quadruplicate and in biological triplicate.

### Immunoblotting After BN-PAGE

Proteins separated on Blue Native PAGE gels (NativePAGE™ 3-12% BisTris Protein Gels, Thermo Fisher Scientific, cat. BN1001BOX) were transferred to PVDF membrane. Membranes were stripped to remove the blue-staining (Restore™ PLUS Western Blot Stripping Buffer, Thermo Fisher Scientific, cat. 46430, 3 min) and probed with anti-OxPhos complex I (39 kDa subunit; clone 20C11, Thermo Fisher Scientific, cat. A21344), anti-SDHA complex II (2E3GC12FB2AE2, Abcam, cat. ab14715), anti-OxPhos complex III (core 1 subunit; clone 16D10, Thermo Fisher Scientific, cat. A21362), anti-OxPhos complex IV (subunit 1; clone 1D6E1A8, Invitrogen cat. 459600), anti-ATPSA complex V (15H4C4, Abcam, cat. ab14748) and Amersham ECL secondary HRP-conjugated antibodies (GE Healthcare). Proteins detection was performed using Clarity™ Western ECL Substrate Detection Reagent (Biorad, cat. 1705061) and the film exposure time was adjusted according to signal intensity.

### Analysis of Mitochondrial Ribosome Profile on Density Gradients

Cells were plated 24 h prior to treatment and incubated with Q/D for further 2 or 48 h. Total cell lysate was prepared by resuspending the cell pellet in lysis buffer (50 mM Tris pH 7.4, 150 mM NaCl, 1 mM EDTA, 1% Triton X-100, EDTA-free Protease Inhibitor Cocktail, Roche), incubated on a roller at 4° C. for 20 min, and centrifuged at 5,000 g for 5 min. The supernatant was then loaded onto a linear sucrose gradient (2 mL 10-30% (v/v) in 50 mM Tris-HCl (pH 7.2), 80 mM NaCl, 20 mM MgCl₂), and centrifuged for 2 h and 15 min at 39,000 rpm at 4° C. (Beckman Coulter TLS-55 rotor). Twenty fractions (100 μL) were collected and 6.5 μL aliquots were analyzed directly by immunoblotting.

### REFERENCES

1. Stupp, R. et al. Radiotherapy plus Concomitant and Adjuvant Temozolomide for Glioblastoma. N. Engl. J. Med. 352, 987-996 (2005)
2. Cavalli, L. R., Varella-Garcia, M. & Liang, B. C. Diminished tumorigenic phenotype after depletion of mitochondrial DNA. Cell Growth Differ. 8, 1189-98 (1997).
3. Tan, A. S. et al. Mitochondrial Genome Acquisition Restores Respiratory Function and Tumorigenic Potential of Cancer Cells without Mitochondrial DNA. Cell Metab. 21, 81-94 (2015)
4. Zu, X. L. & Guppy, M. Cancer metabolism: facts, fantasy, and fiction. Biochem. Biophys. Res. Commun. 313, 459-65 (2004).
5. Janiszewska, M. et al. Imp2 controls oxidative phosphorylation and is crucial for preserving glioblastoma cancer stem cells. Genes Dev. 26, 1926-44 (2012).
6. Vlashi, E. et al. Metabolic state of glioma stem cells and nontumorigenic cells. Proc. Natl. Acad. Sci. U.S.A. 108, 16062-7 (2011).
7. Marziali, G. et al. Metabolic/Proteomic Signature Defines Two Glioblastoma Subtypes With Different Clinical Outcome. Sci. Rep. 6, 21557 (2016).
8. Ropolo, M. et al. Comparative Analysis of DNA Repair in Stem and Nonstem Glioma Cell Cultures. Mol. Cancer Res. 7, 383-392 (2009).
9. Vecchio, D. et al. Predictability, efficacy and safety of radiosensitization of glioblastoma-initiating cells by the ATM inhibitor KU-60019. Int. J. Cancer 135, 479-491 (2014).
10. Vecchio, D. et al. Pharmacokinetics, pharmacodynamics and efficacy on pediatric tumors of the glioma radiosensitizer KU60019. Int. J. Cancer 136, 1445-1457 (2015).
11. Sturm, D. et al. Hotspot Mutations in H3F3A and IDH1 Define Distinct Epigenetic and Biological Subgroups of Glioblastoma. Cancer Cell 22, 425-437 (2012).
12. Pollard, S. M. et al. Glioma Stem Cell Lines Expanded in Adherent Culture Have Tumor-Specific Phenotypes and Are Suitable for Chemical and Genetic Screens. Cell Stem Cell 4, 568-580 (2009).
13. Conti, L. et al. REST Controls Self-Renewal and Tumorigenic Competence of Human Glioblastoma Cells. PLoS One 7, e38486 (2012).
14. Sun, Y. et al. Long-term tripotent differentiation capacity of human neural stem (NS) cells in adherent culture. Mol. Cell. Neurosci. 38, 245-258 (2008).
15. Bacque, E., Barriere, J.-C. & Berthaud, N. Recent Progress in the Field of Antibacterial Pristinamycins. Curr. Med. Chem.-Anti-Infective Agents 4, 185-217 (2005).

1. A compound of formula (I) for use in the treatment of a tumor, 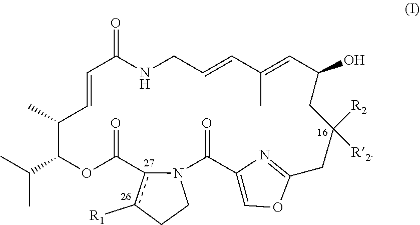
wherein
if the [symbol] bond represents a double bond, then R₁ is H, R₂ is selected from OH, F, NH₂, NHCH₃, N(CH₃)₂, OCF₃, Br, Cl, I, N₃, CN, SCN, and R′₂ is H, or R₂ and R′₂ form together an oxo group;
if the [symbol] bond represents a single bond with H in 27 (27R configuration), then R₁ is H, R₂ is selected from OH, F, NH₂, NHCH₃, N(CH₃)₂, OCF₃, Br, Cl, I, N₃, CN, SCN, and R′₂ is H, or R₂ and R′₂ form together an oxo group;
if the [symbol] bond represents a single bond with H in 27 (27S configuration), then R₁ is SO₂(CH₂)₂N(CH₂CH₃)₂ (26R configuration), R₂ is OH, and R′₂ is H, or R₂ and R′₂ form together an oxo group;
and a pharmaceutically acceptable stereoisomer and/or salt thereof.

2. The compound of formula (I) for use according to claim 1, wherein the compound of formula (I) is in combination with quinupristin or a pharmaceutically acceptable salt thereof.

3. The compound of formula (I) for use according to claim 1, wherein R₂ is selected among OH, F and NHCH₃ or R₂ and R′₂ form together an oxo group.

4. The compound of formula (I) for use according to claim 1, wherein the compound of formula (I) is selected from
(16R)-16-deoxo-16-hydroxypristinamycin II_{A},
(16S)-16-deoxo-16-hydroxypristinamycin II_{A},
(16R)-16-deoxo-16-dalfopristin,
(16S)-16-deoxo-16-dalfopristin,
(16R)-16-deoxo-16-fluoropristinamycin II_{A},
(16S)-16-deoxo-16-fluoropristinamycin II_{A},
(16R)-16-deoxo-16-fluoropristinamycin II_{B},
(16R)-16-Deoxo-16-methylaminopristinamycin II_{A},
(16S)-16-Deoxo-16-methylaminopristinamycin II_{A},
pristinamycin II_{A} and
dalfopristin.

5. The compound of formula (I) for use according to claim 1, wherein the compound of formula (I) is selected from:
(16R)-16-deoxo-16-hydroxypristinamycin II_{A},
(16R)-16-deoxo-16-dalfopristin,
(16R)-16-deoxo-16-fluoropristinamycin II_{A},
(16R)-16-deoxo-16-fluoropristinamycin II_{B},
(16R)-16-Deoxo-16-methylaminopristinamycin II_{A},
(16S)-16-Deoxo-16-methylaminopristinamycin II_{A},
pristinamycin II_{A} and
dalfopristin.

6. The compound of formula (I) for use according to claim 1, wherein the compound of formula (I) is selected from
(16R)-16-deoxo-16-hydroxypristinamycin II_{A},
(16S)-16-deoxo-16-hydroxypristinamycin II_{A},
(16R)-16-deoxo-16-dalfopristin,
(16S)-16-deoxo-16-dalfopristin,
(16R)-16-deoxo-16-fluoropristinamycin II_{A},
(16S)-16-deoxo-16-fluoropristinamycin II_{A}
(16R)-16-deoxo-16-fluoropristinamycin II_{B},
(16R)-16-Deoxo-16-methylaminopristinamycin II_{A},
(16S)-16-Deoxo-16-methylaminopristinamycin II_{A},
pristinamycin II_{A}, and
dalfopristin;
and is in combination with quinupristin or a pharmaceutically acceptable salt thereof.

7. The compound of formula (I) for use according claim 6, wherein the combination contains quinupristin and dalfopristin in a weight ratio equal to 30:70.

8. The compound of formula (I) for use according to claim 1, wherein the tumor is dependent on oxidative phosphorylation.

9. The compound of formula (I) for use according to claim 1, wherein the tumor is selected from glioblastoma multiforme, acute myeloid leukemia, chronic myeloid leukemia, epithelial ovarian cancer, pancreatic ductal adenocarcinoma, colorectal cancer, prostate cancer, melanoma, breast cancer and lung cancer.

10. The compound of formula (I) for use according to claim 1, wherein the tumor is glioblastoma multiforme.

11. The compound of formula (I) for use according to claim 1, wherein the compound of formula (I) is suitable for being administered along with at least one other treatment of the tumor.

12. The compound of formula (I) for use according to claim 11, wherein the at least one other treatment of the tumor is selected from surgery, radiation, and chemotherapy.

13. The compound of formula (I) for use according to claim 1, wherein the compound of formula (I) is suitable for intravenous administration.

14. Pharmaceutical composition for use in the treatment of a tumor comprising:
(a) a compound of formula (I), 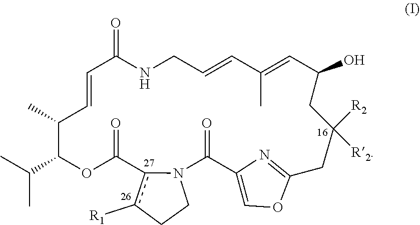
wherein
if the [symbol] bond represents a double bond, then R₁ is H, R₂ is selected from OH, F, NH₂, NHCH₃, N(CH₃)₂, OCF₃, Br, Cl, I, N₃, CN, SCN, and R′₂ is H, or R₂ and R′₂ form together an oxo group;
if the [symbol] bond represents a single bond with H in 27 (27R configuration), then R₁ is H, R₂ is selected from OH, F, NH₂, NHCH₃, N(CH₃)₂, OCF₃, Br, Cl, I, N₃, CN, SCN, and R′₂ is H; or R₂ and R′₂ form together an oxo group;
0 if the [symbol] bond represents a single bond with H in 27 (27S configuration), then R₁ is SO₂(CH₂)₂N(CH₂CH₃)₂ (26R configuration), R₂ is OH, and R′₂ is H, or R₂ and R′₂ form together an oxo group;
and a pharmaceutically acceptable stereoisomer and/or salt thereof; and
(b) at least one pharmaceutically acceptable carrier.

15. The pharmaceutical composition for use according to claim 14, wherein the compound of formula (I) is in combination with quinupristin or a pharmaceutically acceptable salt thereof.
